(19) United States
(12) Patent Application Publication
(10) Pub. No.: US 2020/0087666 A1
(43) Pub. Date: Mar. 19, 2020

(54) COMPOUNDS TARGETING LONG NON CODING RNA FOR THE TREATMENT OF CANCER

(71) Applicant: FUNDACIÓN PARA LA INVESTIGACIÓN MÉDICA APLICADA, Pamplona-Navarra (ES)

(72) Inventors: Maite HUARTE MARTÍNEZ, Pamplona-Navarra (ES); Alejandro ATHIE CUERVO, Pamplona-Navarra (ES)

(21) Appl. No.: 16/130,018

(22) Filed: Sep. 13, 2018

(51) Int. Cl. C12N 15/113; A61P 35/00; A61K 31/7088; C12N 15/11

(52) U.S. Cl. CPC C12N 15/1135; A61P 35/00; C12N 2310/14; C12N 15/111; A61K 31/7088

(57) ABSTRACT

The present invention relates to inhibitors of a non-long coding RNA and to pharmaceutical compositions comprising a non-long coding RNA inhibitor, which may further comprise an additional compound. The invention also relates to methods of treating a tumor and to methods of diagnosing and treating a tumor in a subject by using a long non-coding RNA and its inhibitors, and also to a method for increasing the migration of cytotoxic immune cells towards a tumor.

### FIELD OF THE INVENTION

The present invention relates to the field of cancer therapeutics and, more particularly, to inhibitors of long non-coding RNAs and to methods of treating and diagnosing a tumor in a subject by using a long non-coding RNA and its inhibitors.

### BACKGROUND OF THE INVENTION

Most cancers arise and later progress due to the complex interaction of somatic and germline mutations with various environmental factors. Many of those mutations lie within regions of the genome devoid of protein coding genes however may contain a different type of genes that can exert their functions as RNA molecules, the non-coding RNAs. Most non-coding RNAs are longer than 200 nucleotides and are therefore classified as long non-coding RNAs (lncRNAs). The number of lncRNAs encoded by human cells is large, catalogued in a range that spans from 16,000 genes encoding close to 28,000 transcripts-according to the estimation of GENECODE to more than 60,000 lncRNA transcripts identified across multiple tumor types (Iyer, M. K. et al. 2015. Nat Genet 47(3):199-208). Notably, a number of studies have shown that many lncRNAs are functional molecules that can regulate different aspects of the cell biology through multiple mechanisms. In agreement with this, it has been observed that alterations in lncRNAs are inherent to cancer, and can impact several hallmarks of the disease. However, despite the high number of lncRNAs encoded by the human genome, the understanding of their contribution to the disease still remains poor. Moreover, while identification of relevant cancer-driver lncRNAs is a necessary step towards the comprehension of the mechanisms of tumor progression, it represents a major challenge due to different reasons: (i) A high percentage of the thousands of uncharacterized lncRNAs present altered expression in cancer (Iyer, M. K. et al. 2015. Nat Genet 47(3):199-208) but most of them possibly are passenger alterations. (ii) The high heterogeneity of cancer and the poor knowledge of lncRNA functionality difficult the identification of lncRNA alterations relevant to the disease. In addition, (iii) most studies fail to pinpoint the mechanisms by which lncRNAs may promote oncogenesis, an important requirement prior to the development of lncRNA-centred therapies.

Therefore, it is necessary to identify lncRNAs that can be new targets to inhibit tumor progression.

### SUMMARY OF THE INVENTION

In a first aspect, the invention relates to an inhibitor of functional expression of AC083973.1 gene, wherein the inhibitor is selected from (i) a nucleic acid that specifically binds to the AC083973.1 gene or to the transcriptional product of said gene blocking the expression of said gene and (ii) a nuclease that specifically binds and enzymatically inactivates said gene.

In a second aspect, the invention relates to a pharmaceutical composition comprising an effective amount of the inhibitor according to the first aspect of the invention.

In a third aspect, the invention relates to a method of treating a tumor in a subject comprising administering to the subject a therapeutic effective amount of the inhibitor according to the first aspect of the invention, wherein the tumor is characterized by having increased copy number of the AC083973.1 gene, and/or decreased methylation of said gene and/or increased levels of the transcriptional product of said gene compared to a reference value.

In a fourth aspect, the invention relates to a method of diagnosing and treating a tumor in a subject comprising:

(i) determining the copy number of the AC083973.1 gene, and/or the methylation level of said gene and/or the level of the transcriptional product of said gene in a sample,
(ii) comparing the copy number and/or methylation level and/or level obtained under (i) with a reference value,
(iii) diagnosing the subject as having a tumor susceptible of being treated by an inhibitor according to the invention when the copy number of the gene is increased and/or the methylation level is decreased and/or the level of the transcriptional product of the gene is increased compared to the reference value and
(iv) administering the subject diagnosed with the tumor with a therapeutic effective amount of an inhibitor of functional expression of AC083973.1 according to the first aspect of the invention.

In a fifth aspect, the invention relates to a method for selecting a therapy for treating a subject with a tumor comprising:

(i) determining the copy number of the AC083973.1 gene, and/or the methylation level of said gene and/or the level of the transcriptional product of said gene in a sample,
(ii) comparing the copy number and/or methylation level and/or level obtained under (i) with a reference value,
(iii) selecting a therapy consisting of an inhibitor of functional expression of AC083973.1 according to the first aspect of the invention if the copy number of the gene is increased and/or the methylation level is decreased and/or the level of the transcriptional product of the gene is increased compared to the reference value.

In a sixth aspect, the invention relates to a method for increasing the migration of cytotoxic immune cells towards a tumor comprising administering the inhibitor according to the first aspect of the invention to a subject suffering from said tumor, wherein the tumor has increased copy number of the AC083973.1 gene and/or decreased methylation of said gene and/or increased level of the transcriptional product of said gene compared to a reference value.

### BRIEF DESCRIPTION OF THE FIGURES

FIG. 1A. Overview of cancer-associated SCNAs that contain lncRNAs. Distribution of the TCGA tumor samples and SCNAs detected in the 25 types of cancers.

FIG. 1B. Distribution of SCNAs in the different cancer types, indicating some of the high-confidence known cancer drivers within the alterations.

FIG. 1C. Pipeline for the selection of SCNAs harboring lncRNAs, integrating SCNA detection, gene annotation and expression analysis. The starting point of the analysis is the copy number data from 7448 tumors, in which 1055 recurrent SCNAs were detected. Then these SCNAs were classified based on gene annotation and gene expression analysis.

FIG. 1D. Transcription factors with binding significantly enriched around the TSS of amplified lncRNAs.

FIG. 1E. Circos plot indicating the copy number-altered lncRNAs present in lung adenocarcinomas. Indicated in bold and squared is de lncRNA RP11-231D20.2 selected for the present study.

FIG. 1F. SCNAs selected by the analysis as containing putative cancer-relevant lncRNAs.

FIG. 2A. ALAL-1 is a potential driver of non-small cell lung cancer. Detailed view of the genomic region of the focal peak mapping to RP11-231D20.2/ALAL-1. Each TCGA-LUAD sample with amplification is represented with a horizontal line, and ranked according to the copy number of the segment shown. The focal amplification CAN 623 defined by GISTIC 2.0 algorithm mapping to ALAL-1 locus is indicated in read.

FIG. 2B. Number of tumors presenting amplification of ALAL-1 in different cohorts of lung adenocarcinoma.

FIG. 2C. Expression of ALAL-1 in TCGA-LUAD cohort comparing the samples based on the presence (n=43) or absence (n=322) of the amplification.

FIG. 2D. Expression of ALAL-1 in tumor (n=291) vs normal (n=21) samples from the TCGA-LUAD.

FIG. 2E. Percentage of samples with amplification and expression (FPKM>0) of ALAL-1.

FIG. 2F. Schematic representation of the 5′ of ALAL-1 locus, indicating the two methylated CpGs (cg26394282, cg16230352), the RNA-seq and H3K4Me3 ChIP-seq signals.

FIG. 2G. Methylation level reported using the beta value for the two ALAL-1 associated CpGs in normal (N) and tumor (T) samples from the TCGA-LUAD cohort. Statistical significance is represented as (*) p-value ≤0.05, (**) p-value ≤0.01, (***) p-value ≤0.001.

FIG. 2H. Methylation level reported using the beta value for the two ALAL-1 associated CpGs in normal (N) and tumor (T) samples from the TCGA-LUSC cohort. Statistical significance is represented as (*) p-value ≤0.05, (**) p-value ≤0.01, (***) p-value ≤0.001.

FIG. 3A. ALAL-1 gene structure and isoform expression. Representation of the six different isoforms annotated in ALAL-1 locus with their Ensembl Transcript IDs. Isoforms are represented in the 5′ to 3′ direction and exons are shadowed in grey and identified with letters (A, B, C, D and E). ALAL-1 predominant form (RP11-231D20.2) is highlighted in red.

FIG. 3B. RNA-seq track showing the expression of ALAL-1 in 9 cell lines from the ENCODE project.

FIG. 3C. RNA-seq data from the TCGA-LUAD sample TCGA-44-7661-01A with a mean expression of ALAL-1, supporting the expression of ENST00000521802.

FIG. 3D. Relative expression of ALAL-1 quantified by qRT-PCR with several set of primers mapping to different exons.

FIG. 3E. Expression of ALAL-1 in lung squamous carcinoma (LUSC) tumors and normal samples.

FIG. 3F. Expression of ALAL-1 in head and neck squamous carcinoma (HNSC) tumors and normal samples.

FIG. 3G. ALAL-1 expression quantified by microarray analysis (probe 231378_at) of cohort GSE19188 including 62 adjacent normal lung tissues and 94 NSCLC tumors. Statistical significance is represented as (*) p-value ≤0.05, (**) p-value ≤0.01, (***) p-value ≤0.001.

FIG. 3H. ALAL-1 expression quantified by microarray analysis (probe 231378_at) of cohort GSE18842 with 45 paired samples (normal/tumor) of NSCLC. Statistical significance is represented as (*) p-value ≤0.05, (**) p-value ≤0.01, (***) p-value ≤0.001.

FIG. 3I. ALAL-1 expression quantified by microarray analysis (probe 231378_at) of cohort GSE19804 including 60 paired samples (normal/tumor) from nonsmoking cancer female patients. Statistical significance is represented as (*) p-value ≤0.05, (**) p-value ≤0.01, (***) p-value ≤0.001.

FIG. 4A. ALAL-1 promotes an oncogenic phenotype in lung cancer cells. Amplification and expression (FPKM>0) of ALAL-1 in lung cancer cell lines.

FIG. 4B. Copy number of ALAL-1 locus in different lung cancer cell lines. Data were retrieved from the CCLE.

FIG. 4C. Experimental quantification of the copy number of ALAL-1 locus using qRT-PCR from genomic DNA (gDNA) in different cell lines (BJ [normal] human foreskin fibroblasts, [AD] adenocarcinoma, [SQ] squamous cell carcinoma).

FIG. 4D. ALAL-1 expression in the same cell lines where copy number was estimated.

FIG. 4E. Schematic representation of the CRISPR/Cas9 strategy used to delete exon D of ALAL-1.

FIG. 4F. Relative DNA copy number of ALAL-1 exon D quantified by qRT-PCR using gDNA from the CRISPR/Cas9 clones 23 and 24.

FIG. 4G. Expression of ALAL-1 in clones 23 and 24. RNA levels are represented relative to ALAL-1 expression in HCC95 cells.

FIG. 4H. Cell proliferation determined by MTS assay.

FIG. 4I. Colony formation assay in cell lines where the copy number of ALAL-1 has been reduced. The number of colonies obtained in each condition is represented.

FIG. 4J. Volume of tumors obtained after subcutaneous injection of HCC95 cells engineered with the CRISPR/Cas9 technology in immune-compromised mice.

FIG. 4K. Knockdown efficiency determined by qRT-PCR in HCC95 cells transfected with control siRNA or ALAL-1-targeting siRNAs (1 and 2).

FIG. 4L. Volumes of tumors formed by subcutaneous injection of HCC95 cells in mice after ALAL-1 knockdown.

FIG. 4M. Size of the tumors at the indicated time points in the three experimental conditions tested. Statistical significance is represented as (*) p-value ≤0.05, (**) p-value ≤0.01, (***) p-value ≤0.001.

FIG. 5A. CRISPR/Cas9 targeting of ALAL-1 locus. Crystal Violet (CV) absorbance measuring the colony formation capacity of the CRISPR/Cas9 engineered cells.

FIG. 5B. Percentage of apoptotic cells from the CRISPR/Cas9 clones where ALAL-1 expression was reduced.

FIG. 6A. ALAL-1 is involved in cell proliferation and colony formation. Cell proliferation measured by MTS of HCC95 cells after ALAL-1 inhibition.

FIG. 6B. Crystal Violet absorbance measuring the colony formation capacity in HCC95 cells transfected with siRNAs (1 and 2) targeting ALAL-1 or control siRNA.

FIG. 6C. Percentage of HCC95 apoptotic cells, measured by flow cytometry, after ALAL-1 inhibition.

FIG. 6D. Inhibition levels of ALAL-1 obtained with siRNAs 1 and 2 in H1648 cells. RNA levels are normalized to HPRT and relative to the control siRNA.

FIG. 6E. Cell proliferation measured by MTS of H1648 cells after ALAL-1 inhibition.

FIG. 6F. Crystal Violet absorbance measuring the colony formation capacity in H1648 cells transfected with siRNAs (1 and 2) targeting ALAL-1 or control siRNA. Absorbance values are represented relative to the control.

FIG. 6G. ALAL-1 levels in HCC95 cells transiently transfected with the pcDNA3-ALAL-1 plasmid.

FIG. 6H. Clonogenic assay of HCC95 cells transiently overexpressing ALAL-1.

FIG. 6I. Clonogenic assay of HCC95 cells transiently overexpressing ALAL-1.

FIG. 6J. ALAL-1 levels in H1648 cells transfected with the pcDNA3-ALAL-1 plasmid.

FIG. 6K. Clonogenic assay of H1648 cells transiently overexpressing ALAL-1.

FIG. 6L. Clonogenic assay of H1648 cells transiently overexpressing ALAL-1.

FIG. 6M. Overexpression levels of ALAL-1 in A549 cells transduced with a retrovirus expressing the lncRNA.

FIG. 6N. Colony formation assay of A549 cells overexpressing ALAL-1.

FIG. 6O. Colony formation assay of A549 cells overexpressing ALAL-1.

FIG. 7A. ALAL-1 is a negative regulator of the NF-κB pathway. ChIP-seq signal of p65 in ALAL-1 locus in HUVEC cells treated or and treated with TNFα. Peaks called in p65 ChIP-seq analysis in A549 cell line and the consensus sequences corresponding to p65 binding sites are indicated below.

FIG. 7B. ALAL-1 RNA levels and p65 protein levels determined in HCC95 cells after p65 knockdown.

FIG. 7C. Time course experiment showing the induction of ALAL-1 in HCC95 cells treated with TNFα.

FIG. 7D. Gene ontology of the significantly enriched data sets obtained with the differentially expressed gene list from ALAL-1 knockdown RNAseq analysis.

FIG. 7E. Expression level of genes related to inflammation and immune response after ALAL-1 knockdown and different times of TNFα treatment.

FIG. 8A. ALAL-1 is a regulator of the NF-κB pathway. Percentage of HCC95 apoptotic cells, measured by flow cytometry of annexin V and 7-AAD stained cells, in the presence or absence of TNF-α treatment.

FIG. 8B. Gene ontology analysis of the genes correlated with ALAL-1 expression in TCGA tumor samples.

FIG. 8C. Expression of genes identified by RNA-seq as regulated by ALAL-1 in the clones where ALAL-1 copy number was reduced by deletion with CRISPR (clones 23 and 24) or HCC95 wt cells. The RNA levels are represented relative to HPRT mRNA levels.

FIG. 9A. ALAL-1 regulates the NF-κB pathway through SART3 and USP4. Subcellular localization of ALAL-1 assessed by cell fractionation and

FIG. 9B. Number of fluorescent foci detected by RNA-FISH in cells transfected with control siRNA (siCtrl) or ALAL-1 siRNA (siALAL-1).

FIG. 9C. Dot plot indicating the number of peptides detected of each of the proteins identified as bound to ALAL-1 but not the control RNA in two independent RNA-pulldown experiments.

FIG. 9D. RNA immunoprecipitation of SART3 followed by qRT-PCR of the bound RNAs. Values are represented relative to the IgG control.

FIG. 9E. Barplots represent the percentage of cells with cytoplasmic or nuclear myc-USP4 localization determined by immunofluorescence in HCC95 cells with and without SART3 overexpression and with and without ALAL-1 inhibition.

FIG. 9F. Network showing TAK1/MAP3K7 as the predicted upstream regulator of a set of genes altered after ALAL-1 knockdown (Analysis performed by Ingenuity IPA, Qiagen Inc.).

FIG. 9G. TAK1 K63 ubiquitination level detected by western blotting on anti-TAK1 immunoprecipitates in the indicated experimental conditions. The numbers are the quantification of the ubiquitinated protein relative to the input TAK1, and the total TAK1 levels detected in the inputs are shown in de lower panel.

FIG. 9H. Level of IKKα and IKKβ phosphorylation detected by western blotting in HCC95 transfected with the indicated siRNAs. α-Tubulin is shown as loading control.

FIG. 9I. Relative firefly luciferase expression driven by a NfκB reporter promoter in HCC95 cells transfected with the indicated siRNAs and untreated or treated with TNFα.

FIG. 10A. ALAL-1 regulates the NF-κB pathway through SART3 and USP4. Detection of ALAL-1 and SART3 interaction by western blot. The upper row shows the RNA pulldown using a protein extract of HCC95 cells transfected with myc-SART3 and blotted with the myc-tag antibody (2276, Cell Signaling); the middle row shows the endogenous SART3, and the lower band the biotinylated RNAs (antisense and ALAL-1) used to pulldown SART3.

FIG. 10B. SART3 localization in A549, HCC95 and H1648 cells, identified by western blot using SART3 antibody (ab155765 Abcam). Lamin A/C and GAPDH were used as controls of nuclear and cytoplasmic fractions respectively.

FIG. 10C. Expression of TAK1 in different conditions of ALAL-1 inhibition and TNFα treatment determined by RNAseq in HCC95 cells.

FIG. 11A. ALAL-1 contributes to the immune evasion of non-small cell lung squamous tumors. Expression of ALAL-1 in TCGA-LUSC cohort, classified based on different immune-phenotypes based on gene expression signatures of different infiltration patterns and levels of cytotoxic cells (Tamborero, D. et al. 2018. Clin Cancer Res. doi: 10.1158/1078-0432.CCR-17-3509).

FIG. 11B. ALAL-1 expression and level of PD-1 positive cells in an independent cohort of LUSC tumors.

FIG. 11C. Schematic representation of the migration assay (left) and number of different cell subpopulations from peripheral blood migrated to HCC95 cells transfected with a control siRNA (siCtrl) or siRNA to knockdown ALAL-1 (siALAL-1).

FIG. 11D. Number of CD3⁺/CD4⁺ and CD3⁺/CD4⁻PD1⁺ cells from peripheral blood when co-stimulated with anti-CD3/CD28 and incubated with the conditioned medium of HCC95 cells transfected with the indicated siRNAs and treated with TNFα.

FIG. 11E. Level of secreted INF□ and IL-2 by lymphocytes treated as in (D).

### DETAILED DESCRIPTION OF THE INVENTION

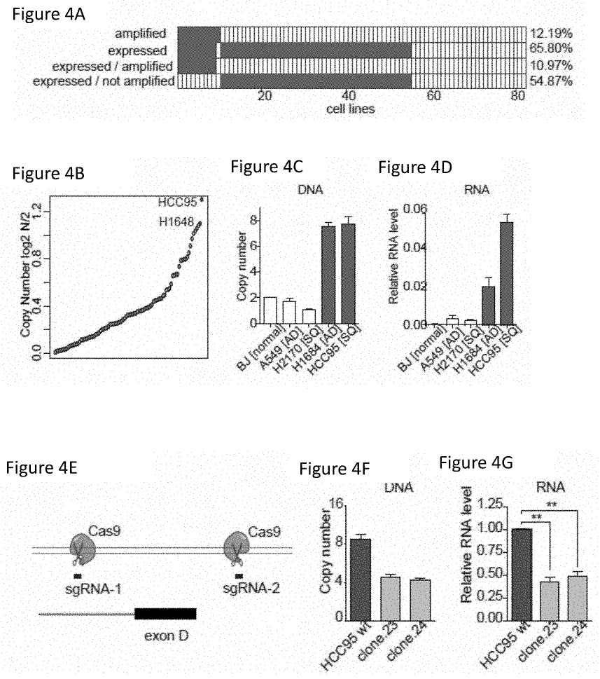

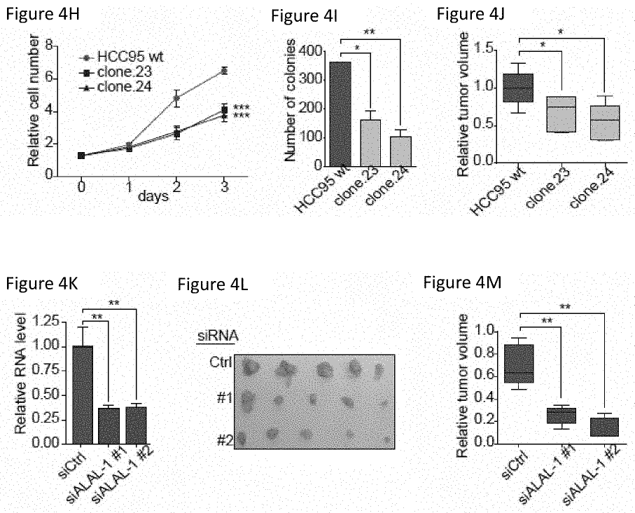

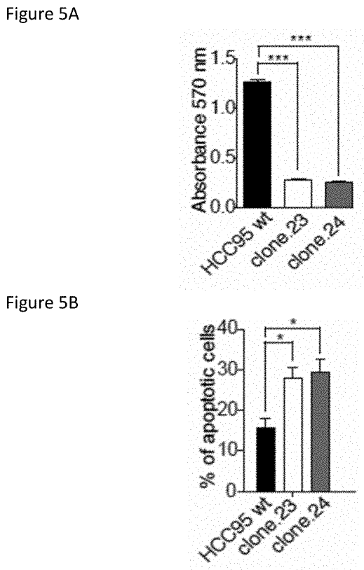

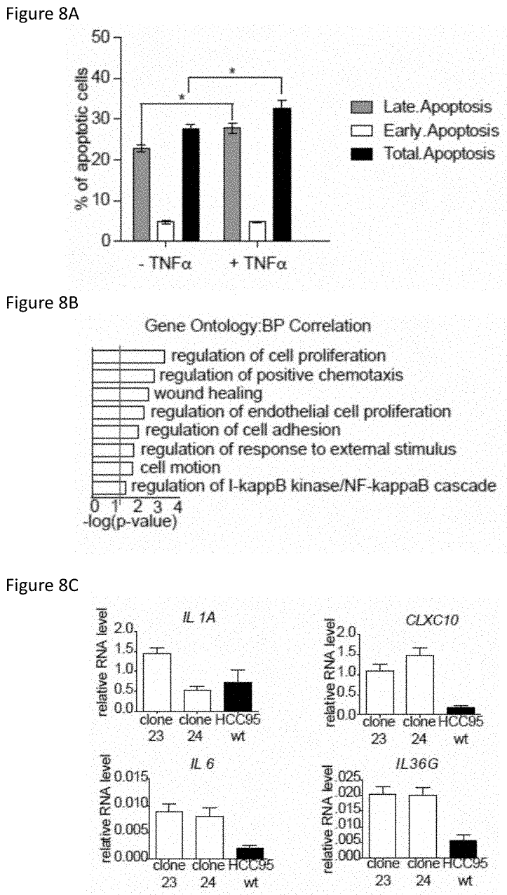

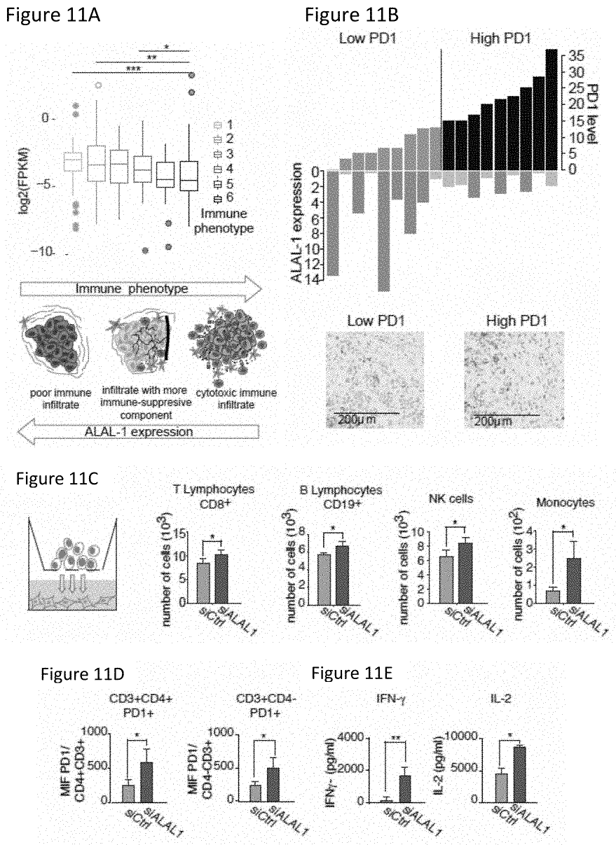

The inventors have developed an inhibitor of functional expression of a long non-coding RNA (lncRNA) for the treatment of cancer. According to the invention, the functional inhibition of the AC083973.1 gene expression reduces the oncogenic phenotype of lung cancer cells, exemplified as reduced in vitro proliferation, reduced colony formation capacity and increased apoptosis, as well as reduced tumor growth capacity in xenograft mouse models, regardless of the inhibition is performed by genomic deletion of the AC083973.1 gene (FIGS. 4F-J and FIG. 5) or by decreasing its RNA levels (FIGS. 8 A-C and FIGS. 4L-M). Additionally, it is shown that inhibition of the lncRNA product of AC083973.1 gene contributes to reduce the immune evasion of non-small cell lung squamous tumors, exemplified as increased migration of CD8+ T lymphocytes, CD19+ B lymphocytes, NK cells and monocytes towards the tumor cells (FIG. 11C).

### Inhibitor of Functional Expression of AC083973.1

In a first aspect, the invention relates to an inhibitor of functional expression of AC083973.1 gene, wherein the inhibitor is selected from (i) a nucleic acid that specifically binds to the AC083973.1 gene or to the transcriptional product of said gene blocking the expression of said gene and (ii) a nuclease that specifically binds and enzymatically inactivates said gene.

As it is used herein, the term “inhibitor of functional expression” is understood as any substance or compound which is capable of specifically silencing, reducing, preventing and/or blocking the expression of the gene, either by preventing the transcription of the gene, therefore avoiding the formation of any of the transcriptional products of the gene, or promoting the degradation of any of the transcriptional products of the gene AC083973.1.

As it is used herein the term “AC083973.1”, also referred by the inventors to as “ALAL-1” or “RP11-231D20.2”, corresponds to the human gene AC083973.1 as identified by the ID ENSG00000253408 in the Ensembl database (according to the release 93 of July 2018). AC083973.1 encodes at least six different transcripts, identified in Ensembl as AC083973.1-206 (ENST00000518213.1, SEQ ID NO: 13), AC083973.1-205 (ENST00000518994.1, SEQ ID NO: 14), AC083973.1-201 (ENST00000523459.5, SEQ ID NO: 15), AC083973.1-203 (ENST00000521802.5, SEQ ID NO: 16), AC083973.1-202 (ENST00000521470.1, SEQ ID NO: 17) and AC083973.1-204 (ENST00000520890.5, SEQ ID NO: 18) (release 93 of July 2018). According to Gencode (release 28 of November 2017), it is also referred as RP11-231D20.2.

In a particular embodiment, the inhibitor of functional expression of AC083973.1 gene is a nucleic acid that specifically binds to the AC083973.1 gene or to the transcriptional product of said gene blocking the expression of said gene.

“Nucleic acids” as used herein mean biopolymers of nucleotides, which are linked with one another via phosphodiester bonds (polynucleotides, polynucleic acids). Nucleic acids may additionally be linked via phosphorothioate bonds when chemically synthesized. Depending on the type of sugar in the nucleotides (ribose or deoxyribose), one distinguishes the two classes of the ribonucleic acids (RNA) and the deoxyribonucleic acids (DNA). As used herein it relates to any natural or non-natural nucleic acid” and “oligonucleotide” and “polynucleotide” are used interchangeably in the context of the present invention. In the context of the present invention “Natural nucleotides” mean nucleotides that can be purified from natural sources. “Non-natural nucleotides” are defined as those produced using recombinant expression systems and, optionally, purified, chemically synthesized, etc. When appropriate, for example, in the case of chemically synthesized molecules, the nucleic acids can comprise nucleoside analogues such as analogues having chemically modified bases or sugars, modifications of the backbone, etc. A nucleic acid sequence is represented in 5′-3′ direction unless indicated otherwise.

The term “transcriptional product” or “transcript”, as used herein, refers to an RNA derived from the transcription of a gene.

In a preferred embodiment the inhibitor of functional expression of AC083973.1 inhibits the expression of AC083973.1 gene by specifically binding to any one, or any combination, of the transcripts selected from: ENST00000518213.1, ENST00000518994.1, ENST00000523459.5, ENST00000521802.5, ENST00000521470.1 and/or ENST00000520890.5. In a more preferred embodiment the inhibitor inhibits AC083973.1 expression by binding to the ENST00000521802.5 transcript, which is the transcript of sequence SEQ ID NO: 16.

Non-limiting examples of nucleic acids that specifically bind to the AC083973.1 gene or to its transcriptional product blocking the expression of this gene include: single guide RNA (sgRNA), DNA polynucleotides that can be transcribed into a sgRNA, small interfering RNA (siRNA), a short hairpin RNA (shRNA), a microRNA (miRNA) and an antisense oligonucleotide.

The term single guide RNA (“sgRNA”) refers to a chimeric non-coding RNA that contains a targeting sequence (crRNA sequence), that specifically recognizes a region of a gene, and a CRISPR enzyme. The crRNA region is a 20-nucleotide sequence that is homologous to a region in the gene of interest and will direct the CRISPR enzyme activity. As disclosed herein, a CRISPR enzyme can be any known in the art, such as Cas9 and Cpf1.

As disclosed herein, CRISPR is a genome editing method. The use of this technology in genome editing is well described in the art, for example in U.S. Pat. No. 8,697,359 and references cited herein. In short, CRISPR is a microbial nuclease system involved in defense against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the better-characterized systems and carries out targeted DNA double-strand break in four sequential steps. First, two noncoding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple genes can be mutated simultaneously simply by using multiple sgRNAs each targeting a different gene. In addition, where two sgRNAs are used flanking a genomic region, the intervening section can be deleted or inverted.

Cas9 is thus the hallmark protein of the type II CRISPR-Cas system, and is a large monomeric DNA nuclease guided to a DNA target sequence adjacent to the PAM (protospacer adjacent motif) sequence motif by a complex of two non-coding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with a sgRNA can introduce site-specific double strand breaks (DSBs) into genomic DNA of live cells from various organisms. For applications in eukaryotic organisms, codon optimized versions of Cas9, which is originally from the bacterium Streptococcus pyogenes, have been used, although other suitable Cas9 orthologues can be used instead, such as Staphylococcus aureus Cas9 (SaCas9), Campylobacter jejuni Cas9 (CjCas9).

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3.

Cas9 expression plasmids for use in the methods of the invention can be constructed as described in the art.

By “crRNA” or CRISPR RNA is meant the sequence of RNA that contains the protospacer element and additional nucleotides that are complementary to the tracrRNA.

By “tracrRNA” (transactivating RNA) is meant the sequence of RNA that hybridises to the crRNA and binds a CRISPR enzyme, such as Cas9 thereby activating the nuclease complex to introduce double-stranded breaks at specific sites within the genomic sequence of at least one of the alpha-, gamma- and/or omega gliadin.

By “protospacer element” is meant the portion of crRNA (or sgRNA) that is complementary to the genomic DNA target sequence, usually around 20 nucleotides in length. This may also be known as a spacer or targeting sequence.

By “sgRNA” (single-guide RNA) is meant the combination of tracrRNA and crRNA in a single RNA molecule, preferably also including a linker loop (that links the tracrRNA and crRNA into a single molecule). “sgRNA” may also be referred to as “gRNA” and in the present context, the terms are interchangeable. The sgRNA or gRNA provide both targeting specificity and scaffolding/binding ability for a Cas nuclease. A gRNA may refer to a dual RNA molecule comprising a crRNA molecule and a tracrRNA molecule.

A sgRNA can be provided as an RNA molecule or as a DNA molecule that is transcribed into the functional sgRNA. In a particular embodiment, DNA polynucleotides of SEQ ID NO: 1 and 2 are hybridized and cloned in a vector that can be administered to a cell and, once transcribed, gives rise to a sgRNA of sequence CACCGUUCCCAGAUGGGGCGACGGC (SEQ ID: 69). In another particular embodiment, DNA polynucleotides of SEQ ID NO: 3 and 4 are hybridized and cloned in a vector that can be administered to a cell and, once transcribed, gives rise to a sgRNA of sequence CACCGUAUAUUGCUAAGACGUUGAC (SEQ ID: 70).

By “CRISPR enzyme” is meant an RNA-guided DNA endonuclease that can associate with the CRISPR system. Specifically, such an enzyme binds to the tracrRNA sequence. In one embodiment, the CRIPSR enzyme is a Cas protein (“CRISPR associated protein), preferably Cas9 or Cpf1, more preferably Cas9.

The term small interfering RNA (“siRNA”) refers to a duplex of small inhibiting RNAs which induce the RNA interference pathway. These molecules can vary in length (generally from 18 to 30 base pairs) and contain variable degrees of complementarity to their target mRNA in the antisense strand. Some siRNAs, but not all, have unpaired overhanging bases at the 5′ or 3′ end of the sense strand and/or the antisense strand. The term “siRNA” includes duplexes of two individual strands. As it is used herein, the siRNA molecules are not limited to RNA molecules but further include nucleic acids with one or more chemically modified nucleotides, such as morpholines.

As it is used herein, the term “shRNA” or “short hairpin RNA” refers to a double stranded RNA (dsRNA) where the two strands are bound by a strand without interrupting the nucleotides between the 3′ end of one strand and the 5′ end of the other respective strand to form a duplex structure. shRNAs can be used to silence a target gene expression via RNA interference since, once the shRNA is processed is located to the RNA-induced silencing complex (RISC), targeting RISC to an mRNA that has a complementary sequence. RISC may cleave this mRNA or repress its translation.

The term “micro RNA” or “miRNA” refers to short single-stranded RNA molecules, typically around 21 to 23 nucleotides long, capable of regulating gene expression. miRNAs may be synthetic (i.e., recombinant) or natural. Natural miRNAs are encoded by genes that are transcribed from DNA and processed from primary transcripts (“pri-miRNA”) to short stem-loop structures (“pre-miRNA”), and finally to mature miRNA. Mature miRNA molecules are partially complementary to one or more mRNA molecules, and downregulate gene expression via a process similar to RNA interference, or by inhibiting translation of mRNA.

As it is used herein, an “antisense sequence” or “antisense oligonucleotides (ASOs)” includes antisense or sense oligonucleotides comprising a single-stranded nucleic acid sequence (RNA or DNA) capable of binding to target mRNA sequences (sense) or DNA sequences (antisense).

In another particular embodiment, the inhibitor of the functional expression of AC083973.1 gene is a nuclease that specifically binds and enzymatically inactivates the AC083973.1 gene.

As disclosed herein a “nuclease” means an enzyme capable of cleaving the phosphodiester bonds between monomers of nucleic acids. They may cause single and/or double stranded breaks in their target molecules. Illustrative non-limitative examples of such nucleases include transcription activator-like effector nucleases (TALEN), zinc-finger nucleases and ribozymes. As it is used herein, the term “transcription activator-like effector nuclease” or “TALEN” refer to restriction enzymes that can be engineered to cut specific sequences of DNA. They are made by fusing a TAL effector DNA-binding domain to a DNA cleavage domain (a nuclease which cuts DNA strands). They can be engineered to bind to practically any desired DNA sequence, so when combined with a nuclease, DNA can be cut at specific locations. The restriction enzymes can be introduced into cells, for use in gene editing or for genome editing in situ, a technique known as genome editing with engineered nucleases.

By “TAL effector” (transcription activator-like (TAL) effector) or TALE is meant a protein sequence that can bind the genomic DNA target sequence (a sequence within at least one of the alpha-, gamma- and/or omega gliadin genes) and that can be fused to the cleavage domain of an endonuclease such as FokI to create TAL effector nucleases or TALENS or meganucleases to create megaTALs. A TALE protein is composed of a central domain that is responsible for DNA binding, a nuclear-localisation signal and a domain that activates target gene transcription. The DNA-binding domain consists of monomers and each monomer can bind one nucleotide in the target nucleotide sequence. Monomers are tandem repeats of 33-35 amino acids, of which the two amino acids located at positions 12 and 13 are highly variable (repeat variable diresidue, RVD). It is the RVDs that are responsible for the recognition of a single specific nucleotide. HD targets cytosine; NI targets adenine, NG targets thymine and NN targets guanine (although NN can also bind to adenine with lower specificity).

As it is used herein the term “zinc-finger nucleases” or “ZFN” refer to artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms.

As it is used herein, the term “ribozyme” or “RNA enzyme” or “catalytic RNA” refers to an RNA molecule which catalyzes a chemical reaction. Ribozymes may be used to hydrolyse phosphodiester bonds in other RNAs. The nucleic acid with the capacity to inhibit the expression of ALAL-1 may contain one or more modifications in the nucleobases, in the sugars, and/or in the bonds between nucleotides.

Modifications to one or more residues of the nucleic acid backbone may comprise one or more of the following: modifications of the sugar at 2′ such as 2′-O-methyl (2′-OMe), 2′-O-methoxyethyl (2′-MOE), 2′-O-methoxyethoxy, 2′-fluoro (2′-F), 2′-allyl, 2′-O-[2-(methylamino)-2-oxoethyl], 2′-O—(N-methylcarbamate); modifications of the sugar at 4′ including 4′-thio, 4′-CH2-O-2′ bridge, 4-(CH2)2-O-2′ bridge; locked nucleic acid (LNA); peptide nucleic acid (PNA); intercalating nucleic acid (INA); twisted intercalating nucleic acid (TINA); hexitol nucleic acids (HNA); arabinonucleic acid (ANA); cyclohexene nucleic acids (CNAs); cyclohexenyl nucleic acid (CeNA); threose nucleic acid (TNA); morpholine oligonucleotides; Gapmers; Mixmers; incorporation of arginine-rich peptides; addition of 5′-phosphate to synthetic RNAs; RNA aptamers (Que-Gewirth NS, Gene Ther. 2007 February; 14(4):283-91); antidote-controlled RNA aptamers in the subject of the specific RNA aptamer (ref. Oney S, Oligonucleotides. 2007 Fall; 17(3):265-74), or any combination thereof.

Modifications to one or more nucleoside bonds of the nucleic acids may comprise one or more of the following: phosphorothioate, phosphoramidate, phosphorodiamidate, phosphorodithioate, phosphoroselenoate, phosphorodiselenoate, phosphoroanilothioate, and phosphoranilidate, or any combination thereof.

A locked nucleic acid (LNA), commonly referred to as inaccessible RNA, is a modified RNA nucleotide. The ribose group of an LNA nucleotide is modified with an extra bridge joining carbons 2′ and 4′ (O2′,C4′-methylene bridge). The bridge “locks” the ribose in the 3′-endo structural conformation, which is usually found in the A form of DNA or RNA. LNA nucleotides can be mixed with DNA or RNA bases in the nucleic acid when desired. Such oligomers are commercially available.

A peptide nucleic acid (PNA) is an artificially synthesized polymer the backbone of which is made up of repeating units of N-(2-aminoethyl)-glycine linked by peptide bonds. The different purine and pyrimidine bases are linked to the backbone by methylene-carbonyl bonds.

An intercalating nucleic acid (INA) is a modified nucleic acid analog comprising normal deoxyribonucleotides covalently bound to hydrophobic insertions. Hexitol nucleic acids (HNAs) are nucleotides formed by natural nucleobases and a phosphorylated 1,5-anhydrohexitol backbone. The molecular associations between HNA and RNA are more stable than that between HNA and DNA and between natural nucleic acids (dsDNA, dsRNA, DNA/RNA). Other synthetically modified oligonucleotides comprise ANA (arabinonucleic acid), CNAs (cyclohexene nucleic acids), CeNA (cyclohenexyl nucleic acid), and TNA (threose nucleic acid).

Morpholines are synthetic molecules which are the product of redesigning the natural nucleic acid structure. Structurally, the difference between morpholines and DNA or RNA is that while morpholines have standard nucleobases, those bases are linked to 6-membered morpholine rings instead of to deoxyribose/ribose rings, and the non-ionic phosphorodiamidate bonds between the subunits replace the anionic phosphodiester bonds. Morpholines are sometimes referred to as PMO (phosphorodiamidate morpholino oligonucleotide). The 6 membered morpholine ring has the chemical formula O (CH2 CH2)2 NH.

Gapmers or “oligomeric compounds with gaps” are RNA-DNA-RNA chimeric oligonucleotide probes, where DNA windows or gaps are inserted in either a normal or modified RNA oligonucleotide known as “wings”. This modification increases the stability of the oligonucleotide in vivo and makes the probe more prone to interacting with the target, such that shorter probes can be effectively used. Preferably, the wings are modified 2′-O-methyl (OMe) or 2′-O-methoxyethyl (MOE) oligonucleotides which protect the internal block from nuclease degradation. Furthermore, the nucleotides forming the gap or wings can be linked by phosphodiester bonds or by phosphorothioate bonds, which thereby makes them resistant to RNAse degradation. Furthermore, the nucleotides forming the wings can also be modified by the incorporation of bases linked by 3′-methylphosphonate bonds.

Inhibitors suitable for use in the present invention and acting by means of inhibiting the expression of the gene or genes encoding any one of the gene products encoded by AC083973.1 include all those causing a reduction in the mRNA levels of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% with respect to a reference value.

“Reference value”, as used herein relates to a laboratory value used as a reference for the values/data obtained from samples. The reference value (or reference level) can be an absolute value, a relative value, a value which has an upper and/or lower limit, a series of values, an average value, a median, a mean value, or a value expressed by reference to a control or reference value. A reference value can be based on the value obtained from an individual sample, such as, for example, a value obtained from a sample of study but obtained at a previous point in time. The reference value can be based on a high number of samples, such as the values obtained in a population of samples or based on a pool of samples including or excluding the sample to be tested. In a particular embodiment, the reference value is the level of the mRNA transcribed from the AC083973.1 gene in the absence of the inhibitor of the invention.

In a preferred embodiment, the inhibitor inhibits the expression of any one, or any combination, of the transcripts selected from: ENST00000518213.1, ENST00000518994.1, ENST00000523459.5, ENST00000521802.5, ENST00000521470.1 and/or ENST00000520890.5. In a more preferred embodiment the inhibitor inhibits ENST00000521802.5 (SEQ ID NO: 16) expression.

Methods suitable for determining if an inhibitor is capable of inhibiting or blocking the functional expression of AC083973.1 gene include, without limitation, standard assays for determining mRNA expression levels such as qPCR, RT-PCR, RNA protection analysis, Northern blot, RNA dot blot, in situ hybridization, microarray technology, tag based methods such as serial analysis of gene expression (SAGE) including variants such as LongSAGE and SuperSAGE, microarrays, fluorescence in situ hybridization (FISH), including variants such as Flow-FISH, qFiSH and double fusion FISH (D-FISH), and the like.

“Blocking the expression” as used herein means a reduction in the expression or transcription of any one of the transcriptional products of AC083973.1 in relation to a reference value. The reduction can be of at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% with respect to a reference value. In a preferred embodiment the inhibitor inhibits any one, or any combination, of the variants selected from: ENST00000518213.1, ENST00000518994.1, ENST00000523459.5, ENST00000521802.5, ENST00000521470.1 and/or ENST00000520890.5 expression. In a more preferred embodiment the inhibitor inhibits ENST00000521802.5 (SEQ ID NO: 16) expression.

In a preferred embodiment, the inhibitor of functional expression of AC083973.1 is selected from sgRNA, a DNA polynucleotide that can be transcribed into a sgRNA, siRNA, shRNA, miRNA, an antisense oligonucleotide, and/or a ribozyme. In a more preferred embodiment, the inhibitor is selected from sgRNA and/or siRNA. In a more preferred embodiment, the inhibitor is selected from sgRNA sequence SEQ ID NO: 69 and SEQ ID NO: 70. In an even more preferred embodiment, the inhibitor comprises both sgRNA sequence SEQ ID NO: 69 and SEQ ID NO: 70. In another more preferred embodiment, the inhibitor is selected from the DNA polynucleotide that can be transcribed into a sgRNA selected from SEQ ID NO: 1 and SEQ ID NO: 3. In another even more preferred embodiment, the inhibitor comprises both DNA polynucleotides that can be transcribed into a sgRNA of sequences SEQ ID NO: 1 and SEQ ID NO: 3. In another more preferred embodiment, the inhibitor is selected from siRNA sequence SEQ ID NO: 10 and SEQ ID NO: 11.

As disclosed herein to “enzymatically inactivate a gene” means to cleave by means of an enzyme any phosphodiester bond between monomers of nucleic acids such that the gene encoded cannot be replicated, transcribed or expressed in any way.

### Pharmaceutical Compositions

In another aspect the invention relates to a pharmaceutical composition comprising an effective amount of the inhibitor of functional expression of AC083973.1 according to the invention.

In a particular embodiment the pharmaceutical composition of the invention comprises a combination of at least two inhibitors of functional expression of AC083973.1 according to the invention, e.g. two sgRNAs. In a more particular embodiment, the pharmaceutical composition comprises sgRNAs of sequences SEQ ID NO: 69 and SEQ ID NO: 70; or the DNA polynucleotides of sequences SEQ ID NO: 1 and SEQ ID NO: 3 that can be transcribed into sgRNAs.

The inhibitor according to the present invention can be part of a pharmaceutical composition containing a vehicle suitable for the administration thereof to a subject, such that the inhibitor of functional expression of AC083973.1 will be administered to a subject in a pharmaceutical dosage form suitable to that end and will include at least one pharmaceutically acceptable vehicle. Therefore, in a particular embodiment, the inhibitor will be part of a pharmaceutical composition comprising, in addition to the inhibitor of functional expression of AC083973.1 as an active ingredient, at least one vehicle, preferably a pharmaceutically acceptable vehicle. The term “vehicle” generally includes any diluent or excipient with which an active ingredient is administered. Preferably, said vehicle is a pharmaceutically acceptable vehicle for the administration thereof to a subject, i.e., it is a vehicle (e.g., an excipient) approved by a regulatory agency, for example, the European Medicines Agency (EMA), the United States Food & Drug Administration (FDA), etc., or are included in a generally recognized pharmacopeia (e.g., the European Pharmacopeia, the United States Pharmacopeia, etc.) for use in animals, and more particularly in human beings.

The inhibitor of functional expression of AC083973.1 can be dissolved for administration in any suitable medium. Non-limiting illustrative examples of media in which the active ingredient can be dissolved, suspended, or with which they can form emulsions, include: water, ethanol, water-ethanol or water-propylene glycol mixtures, etc., oils, including oils derived from petroleum, animal oils, vegetable oils, or synthetic oils, such as peanut oil, soybean oil, mineral oil, sesame oil, etc., organic solvents such as: acetone, methyl alcohol, ethyl alcohol, ethylene glycol, propylene glycol, glycerin, diethyl ester, chloroform, benzene, toluene, xylene, ethylbenzene, pentane, hexane, cyclohexane, tetrahydrofuran, carbon tetrachloride, chloroform, methylene chloride, trichloroethylene, perchloroethylene, dimethylsulfoxide (DMSO).

Likewise, solid form preparations of the pharmaceutical composition intended for being converted, right before use, into liquid form preparations for oral or parenteral administration, are included. Liquid forms of this type include solutions, suspensions, and emulsions. A review of the different pharmaceutical dosage forms of active ingredients, of the vehicles to be used, and of the manufacturing methods thereof can be found, for example, in the Tratado de Farmacia Galénica, C. Faulí i Trillo, Luzán 5, S. A. de Ediciones, 1993 and in Remington's Pharmaceutical Sciences (A. R. Gennaro, Ed.), 20^{th} edition, Williams & Wilkins PA, USA (2000).

In a non-limiting manner, the administration routes for the inhibitor of functional expression of AC083973.1 include, among others, non-invasive pharmacological administration routes, such as the oral, gastroenteric, nasal, or sublingual route, and invasive administration routes, such as the parenteral route. In a particular embodiment, the inhibitor of functional expression of AC083973.1 is administered in a pharmaceutical dosage form by means of a parenteral route (e.g., intradermal, intramuscular, intraperitoneal, intravenous, subcutaneous, intrathecal, etc.). “Administration by means of a parenteral route” is understood as that administration route consisting of administering the compounds of interest by means of an injection, therefore requiring the use of a syringe and needle. There are different types of parenteral puncture according to the tissue the needle reaches: intramuscular (the compound is injected into the muscle tissue), intravenous (the compound is injected into the vein), subcutaneous (injected under the skin), and intradermal (injected between the layers of skin). The intrathecal route is used for administering into the central nervous system drugs which do not penetrate the blood-brain barrier well, such that the drug is administered into the space surrounding the spinal cord (intrathecal space). The intratumoral administration means the administration of a drug within or introduced directly into a tumor. In a preferred embodiment, the administration is an intradermal, intramuscular, intraperitoneal, intravenous, subcutaneous, intratumoral or intrathecal administration. In a more preferred embodiment the administration is intravenous and/or intratumoral.

As used herein, the term “an effective amount of the inhibitor” means an amount able to reduce the functional expression of AC083973.1 gene at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% with respect to a reference value. In another preferred embodiment, the effective amount of the inhibitor inhibits the expression any one, or any combination, of the transcripts: ENST00000518213.1, ENST00000518994.1, ENST00000523459.5, ENST00000521802.5, ENST00000521470.1 and/or ENST00000520890.5. In a more preferred embodiment the inhibitor inhibits ENST00000521802.5 (SEQ ID NO: 16) expression.

According to the invention, the pharmaceutical composition can further comprise one or more additional compounds. In a preferred embodiment, the compound can be any inhibitor of the immune checkpoint. The term “immune checkpoint inhibitor” as used herein refers to any binding agent or compound that totally or partially inhibit, interfere with or modulate one or more immune checkpoint proteins, such as programmed cell death-1/programmed cell death ligand-1 (PD-1/PD-L1), cytotoxic T-lymphocyte antigen-4 (CTLA-4), indoleamine 2,3-dioxygenase (IDO), T-cell membrane protein-3 (TIM3), lymphocyte activation gene-3 (LAG3), T-cell immunoreceptor with Ig and immunoreceptor tyrosine-based inhibitory motif (ITIM) domains (TIGIT), B- and T-lymphocyte attenuator (BTLA), V-domain Ig suppressor of T-cell activation (VISTA), inducible T-cell COStimulator (ICOS), killer immunoglobulin-like receptors (KIRs), or CD39.

Non limiting examples of immune checkpoint inhibitors include the ones shown in table 1 below:

TABLE 1
Examples of immune checkpoint inhibitors

| | CAS Registry | | |
|---|---|---|---|
| Name | number | Other Names | Target |
| pembrolizumab | 1374853-91-4 | lambrolizumab, Keytruda | PD-1/PD-L1 |
| nivolumab | 946414-94-4 | Opdivo | PD-1/PD-L1 |
| atezolizumab | 1380723-44-3 | Tecentriq, RG7446 | PD-1/PD-L1 |
| avelumab | 1537032-82-8 | Bavencio | PD-1/PD-L1 |
| durvalumab | 1428935-60-7 | Imfinzi, MEDI-4736 | PD-1/PD-L1 |
| ipilimumab | 477202-00-9 | MDX-101, MDX-010 | CTLA-4 |
| tremelimumab | 745013-59-6 | ticilimumab | CTLA-4 |
| spartalizumab | 1935694-88-4 | PDR001 | PD-1/PD-L1 |
| pidilizumab | 1036730-42-3 | CT-011 | PD-1/PD-L1 |
| dostarlimab | 2022215-59-2 | TSR-042 | PD-1/PD-L1 |
| cemiplimab | 1801342-60-8 | REGN2810, SAR439684 | PD-1/PD-L1 |
| SHR-1210 | 1923896-09-6 | INCSHR-1210 | PD-1/PD-L1 |
| LY3300054 | 2161409-32-9 | LY3300054 (Lilly) | PD-1/PD-L1 |
| CK-301 | 2216751-26-5 | CK-301 | PD-1/PD-L1 |
| BMS-936559 | 1422185-22-5 | BMS-936559, | PD-1/PD-L1 |
| | | MDX-1105 | |
| 3D-2-02-0015 | 2102192-68-5 | 3D-2-02-0015; KN035 | PD-1/PD-L1 |
| | | (3D Medicines) | |
| STI-1014 | 1923896-20-1 | STI-1014; STIA 1014 | PD-1/PD-L1 |
| Indoximod | 110117-83-4 | NLG 8189 | IDO |
| epacadostat | 1204669-58-8 | INCB 024360 | IDO |
| BMS-986205 | 1923833-60-6 | (R)-N-(4- | IDO |
| | | chlorophenyl)-2- | |
| | | ((1S,4S)-4-(6- | |
| | | fluoroquinolin-4- | |
| | | yl)cyclohexyl)- | |
| | | propenamide, ONO-7701 | |
| navoximod | 1402837-78-8 | | IDO |
| free base | | | |
| navoximod | 1793075-63-4 | | IDO |
| phosphate | | | |
| 3-(5-fluoro- | 198474-05-4 | EOS200271; | IDO |
| 1H-indol-3- | | PF-06840003 | |
| yl)pyrrolidine- | | | |
| 2,5-dione | | | |
| LY3321367 | 2222123-81-9 | LY3321367 | TIM3 |
| TSR-022 | 2176443-32-4 | TSR-022 | TIM3 |
| OREG-103 | 2179360-61-1 | OREG-103/BY40, | CD39 |
| | | BY40/OREG-103 | |
| MEDI-570 | 2179209-59-5 | MEDI-570 | ICOS |
| GSK3359609 | 2225941-00-2 | GSK3359609 | ICOS |
| JTX-2011 | 2039148-04-2 | JTX-2011 | ICOS |
| lirilumab | 1000676-41-4 | IPH2102, BMS-986015 | KIRs |
| LAG525 | 2072080-18-1 | LAG525, IMP-701 | LAG3 |
| relatlimab | 1673516-98-7 | BMS-986016, | LAG3 |
| | | ONO-4482 | |
| TSR-033 | 2225940-96-3 | TSR-033 | LAG3 |
| IMP321 | 950997-37-2 | IMP321 | LAG3 |
| REGN3767 | 2126132-98-5 | REGN3767 | LAG3 |
| GSK2831781 | 2088304-32-7 | GSK2831781 | LAG3 |
| KD033 | 1923896-14-3 | KD033 | PD-L1 |
| RG6058 | 2138887-88-2 | RG6058 | Tigit |
| OMP-313M32 | 2044984-83-8 | OMP-313M32 | Tigit |
| PF-06688992 | | PF-06688992 (Pfizer) | PD-1/PD-L1 |
| | | (NCT03159117) | |
| FAZ053 | | FAZ053 (Novartis) | PD-1/PD-L1 |
| HTI-1316 | | HTI-1316 | PD-1/PD-L1 |
| KHK2455 | | KHK2455 (Kyowa Kirin) | IDO |
| STI-600 | | STI-600 | TIM3 |
| ENUM005 | | ENUM005 | TIM3 |
| MK-4280 | | MK-4280 | LAG3 |
| BI 754111 | | BI 754111 | LAG3 |
| BMS-986207 | | BMS-986207, | Tigit |
| | | ONO-4686 | |
| AB154 | | AB154 | Tigit |

In a preferred embodiment the immune checkpoint inhibitor is a CTL4-inhibitor. In another preferred embodiment the immune checkpoint inhibitor is a PD-1-inhibitor. In another preferred embodiment the immune checkpoint inhibitor is a PD-L1-inhibitor. In another preferred embodiment the immune checkpoint inhibitor is a combination of a CTL4-inhibitor, a PD-1-inhibitor and/or a PD-L1-inhibitor.

The term “CTLA-4 inhibitor”, as used herein, refers to any compound able to inhibit the function of CTL-4 (also known as cytotoxic T-lymphocyte antigen 4 or CD152). CTLA-4 is a trans-membrane protein found on the surface of T cells, which, when bound to B7 on antigen-presenting cells, prevents T cell activation. The overall effect is immune down-regulation; thus CTLA-4 is described as an immune checkpoint “off switch”.

The term “PD-1 inhibitor”, as used herein, refers to any compound able to inhibit the function of PD-1 (also known as programmed T cell death 1) is a trans-membrane protein found on the surface of T cells, which, when bound to programmed T cell death ligand 1 (PD-L1) on tumor cells, results in suppression of T cell activity and reduction of T cell-mediated cytotoxicity. Thus, PD-1 and PD-L1 are immune down-regulators or immune checkpoint “off switches”. The term “PD-L1 inhibitor”, as used herein, refers to any compound able to inhibit the function of PD-L1.

In an embodiment, optionally in combination with one or more features of the various embodiments described above or below throughout all the description, the immune checkpoint inhibitor of the combination of the invention is suitable to act against immune checkpoint proteins selected from the group consisting of PD-1/PD-L1, CTLA-4, IDO, TIM3, LAG3, TIGIT, BTLA, VISTA, ICOS, KIRs, CD39, and combinations thereof.

In an embodiment, optionally in combination with one or more features of the various embodiments described above or below throughout all the description, the immune checkpoint inhibitors of the combination of the invention is selected from the group consisting of pembrolizumab, nivolumab, atezolizumab, avelumab, durvalumab, ipilimumab, tremelimumab, spartalizumab, pidilizumab, dostarlimab, cemiplimab, SHR-1210, LY3300054, CK-301, BMS-936559, 3D-2-02-0015, STI-1014, Indoximod, epacadostat, BMS-986205, navoximod free base, navoximod phosphate, 3-(5-fluoro-1H-indol-3-yl)pyrrolidine-2,5-dione, LY3321367, TSR-022, OREG-103, MEDI-570, GSK3359609, JTX-2011, lirilumab, LAG525, relatlimab, TSR-033, IMP321, REGN3767, GSK2831781, KD033, RG6058, OMP-313M32, PF-06688992, FAZ053, HTI-1316, KHK2455, STI-600, ENUM005, MK-4280, BI 754111, BMS-986207, and AB154. More particularly, the immune checkpoint inhibitors of the combination of the invention is selected from the group consisting of pembrolizumab, nivolumab, atezolizumab, avelumab, durvalumab, ipilimumab, tremelimumab, spartalizumab, pidilizumab, dostarlimab, cemiplimab, SHR-1210, LY3300054, CK-301, BMS-936559, 3D-2-02-0015, STI-1014, Indoximod, epacadostat, BMS-986205, navoximod free base, navoximod phosphate, 3-(5-fluoro-1H-indol-3-yl)pyrrolidine-2,5-dione, LY3321367, TSR-022, OREG-103, MEDI-570, GSK3359609, JTX-2011, lirilumab, LAG525, relatlimab, TSR-033, IMP321, REGN3767, GSK2831781, KD033, RG6058, and OMP-313M32.

All the terms and embodiments previously described are equally applicable to this aspect of the invention.

### Method of Treating a Tumor

In another aspect the invention relates to a method of treating a tumor in a subject comprising administering to the subject a therapeutic effective amount of the inhibitor according to the invention, wherein the tumor is characterized by having increased copy number of the AC083973.1 gene, and/or decreased methylation of the said gene and/or increased levels of the transcriptional product of said gene compared to a reference value.

Alternatively, in another aspect, the invention relates to the inhibitor of functional expression of AC083973.1 according to the present invention for use as a medicament. In another aspect, the invention relates to an inhibitor of functional expression of AC083973.1 according to the present invention for use in the treatment of a tumor characterized by having increased copy number of the AC083973.1 gene, and/or decreased methylation of the said gene and/or increased levels of the transcriptional product of said gene compared to a reference value.

Alternatively, in another aspect, the invention relates to a use of the inhibitor of the functional expression of AC083973.1 according to the present invention for manufacturing a medicament. In another aspect, the invention relates to the use of the inhibitor of functional expression of AC083973.1 according to the present invention for manufacturing a medicament for treating a tumor characterized by having increased copy number of the AC083973.1 gene, and/or decreased methylation of the said gene and/or increased levels of the transcriptional product of said gene compared to a reference value.

As used herein, the terms “treatment,” “treating,” and the like, comprises any type of therapy, which aims at terminating, preventing, ameliorating and/or reducing the susceptibility to a clinical condition as described herein. Thus, “treatment” “treating” and the like, as used herein, refer to obtaining a desired pharmacologic and/or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. It covers any treatment of a disease in a mammal, particularly in a human, and includes: (a) increasing survival time; (b) decreasing the risk of death due to the disease; (c) preventing the disease from occurring in a subject which may be predisposed to the disease but has not yet been diagnosed as having it; (d) inhibiting the disease, i.e., arresting its development (e.g., reducing the rate of disease progression); and (e) relieving the disease, i.e., causing regression of the disease.

As disclosed herein, the terms “cancer” and “tumor” relate to the physiological condition in mammals characterized by unregulated cell growth. Examples of cancers include, but are not limited to, cancer of the adrenal gland, bone, brain, breast, bronchi, colon and/or rectum, gallbladder, gastrointestinal tract, head and neck, kidneys, larynx, liver, lung, neural tissue, pancreas, prostate, parathyroid, skin, stomach, and thyroid. Other examples of cancers include, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myelodisplastic syndrome, myeloma, mycosis fungoides, neuroblastoma, osteosarcoma, osteogenic and other sarcoma, ovarian tumor, pheochromocytoma, polycythermia vera, primary brain tumor, small-cell lung tumor, squamous cell carcinoma of both ulcerating and papillary type, seminoma, soft tissue sarcoma, retinoblastoma, rhabdomyosarcoma, renal cell tumor or renal cell carcinoma, veticulum cell sarcoma, and Wilm's tumor. Examples of cancers also include astrocytoma, a gastrointestinal stromal tumor (GIST), a glioma or glioblastoma, renal cell carcinoma (RCC), hepatocellular carcinoma (HCC), and a pancreatic neuroendocrine cancer.

In a particular embodiment, the tumor is a solid tumor. The term “solid tumor”, as used herein, refers to an abnormal mass of tissue that usually does not contain cysts or liquid areas. In general, cancer can be divided into solid tumor cancers and hematological cancers. Therefore, all cancer types listed above that are not hematological cancers fall beyond the term “solid tumor” as used herein.

In a preferred embodiment the tumor is lung cancer and/or head and neck cancer. In a more preferred embodiment the lung cancer is non-small cell lung cancer. In a more preferred embodiment the non-small cell lung cancer is squamous cell carcinoma or adenocarcinoma. In another more preferred embodiment the head and neck cancer is head and neck squamous cell carcinoma.

The term “lung cancer” as used herein refers to a malignant lung tumor characterized by uncontrolled cell growth in tissues of the lung. This growth can spread beyond the lung by the process of metastasis into nearby tissue or other parts of the body. Most cancers that start in the lung, known as primary lung cancers, are carcinomas. The two main types are small-cell lung carcinoma (SCLC, characterized by cells that appear small and round under the microscope) and non-small-cell lung carcinoma (NSCLC, characterized by cells that appear larger in size under the microscope). The most common types of NSCLC are squamous cell carcinoma, large cell carcinoma, and adenocarcinoma.

The term “squamous cell carcinoma” as used herein refers to a carcinoma that begins in squamous cells. Squamous cells are thin, flat cells that look like fish scales, and are found in the tissue that forms the surface of the skin, the lining of the hollow organs of the body, and the lining of the respiratory and digestive tracts.

The term “adenocarcinoma” as used herein refers to cancer that begins in glandular (secretory) cells. Glandular cells are found in tissue that lines certain internal organs and makes and releases substances in the body, such as mucus, digestive juices, or other fluids.

The term “head and neck cancer” as used herein refers to cancer that arises in the head or neck region (in the nasal cavity, sinuses, lips, mouth, salivary glands, throat, or larynx).

The term “head and neck squamous cell carcinoma” as used herein refers to cancer of the head and neck that begins in squamous cells (thin, flat cells that form the surface of the skin, eyes, various internal organs, and the lining of hollow organs and ducts of some glands). Squamous cell carcinoma of the head and neck includes cancers of the nasal cavity, sinuses, lips, mouth, salivary glands, throat, and larynx (voice box). Most head and neck cancers are squamous cell carcinomas.

In various embodiments, the patient's cancer treated is a metastatic cancer or a refractory and/or relapsed cancer that is refractory to first, second, or third line treatments. In another embodiment, the treatment is a first, a second, or a third line treatment. As used herein, the phrase “first line” or “second line” or “third line” refers to the order of treatment received by a patient. First line treatment regimens are treatments given first, whereas second or third line treatment are given after the first line therapy or after the second line treatment, respectively. Therefore, first line treatment is the first treatment for a disease or condition. In patients with cancer, primary treatment can be surgery, chemotherapy, radiation therapy, or a combination of these therapies. First line treatment is also referred to those skilled in the art as primary therapy or primary treatment. Typically, a patient is given a subsequent chemotherapy regimen because the patient did not show a positive clinical or only showed a sub-clinical response to the first line therapy, or the first line treatment has stopped. In this context, “chemotherapy” is used in its broadest sense to incorporate not only classic cytotoxic chemotherapy but also molecularly targeted therapies and immunotherapies.

The term “subject” or “patient”, as used herein, refers to all animals classified as mammals. The mammals of preferred embodiments are humans.

As it is used herein, the term “therapeutic effective amount” is understood as any amount of the inhibitor according to the invention able to inhibit the expression of the AC083973.1 gene. In a preferred embodiment, the gene product levels are reduced at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% with respect to a reference value. In another preferred embodiment the inhibitor inhibits the expression of any one, or any combination, of the transcripts ENST00000518213.1, ENST00000518994.1, ENST00000523459.5, ENST00000521802.5, ENST00000521470.1 and/or ENST00000520890.5. In a more preferred embodiment the inhibitor inhibits ENST00000521802.5 (SEQ ID NO: 16) expression.

One skilled in the art can readily determine the therapeutic effective amount of the inhibitor of the invention to be administered to a given subject, by taking into account factors such as the size and weight of the subject; the extent of the disease penetration; the age, health and sex of the subject; the route of administration; and whether the administration is regional or systemic. In a particular embodiment, the therapeutic effective amount of the inhibitor according to the invention, preferably an siRNA, is an amount that when administered to the subject an intracellular concentration of from about 1 nanomolar (nM) to about 100 nM, preferably from about 2 nM to about 50 nM, more preferably from about 20 nM to about 40 nM is achieved in tumor cells In a more particular embodiment the therapeutic effective amount of the inhibitor according to the invention, preferably an siRNA, is an amount that when administered to a subject an intracellular concentration of 30 nM is achieved in the tumor cells. It is contemplated that greater or lesser amounts of the inhibitor can be administered. The skilled person knows how to determine the amount that has to be administered to a subject to achieved a particular intracellular concentration of the inhibitor of the invention.

In a preferred embodiment the inhibitor of the invention to be administered to a given subject is selected from the group: sgRNA, a DNA polynucleotide that can be transcribed into a sgRNA, siRNA, shRNA, miRNA, an antisense oligonucleotide, and/or a ribozyme. In a more preferred embodiment, the inhibitor is selected from sgRNA and/or siRNA. In a still more preferred embodiment, the inhibitor is selected from sgRNA sequence SEQ ID NO: 69 and SEQ ID NO: 70. In an even more preferred embodiment, the inhibitor comprises both sgRNA of sequences SEQ ID NO: 69 and SEQ ID NO: 70. In another more preferred embodiment, the inhibitor is selected from a DNA polynucleotide that can be transcribed into a sgRNA selected from SEQ ID NO: 1 and SEQ ID NO: 3. In another even more preferred embodiment, the inhibitor is comprises both DNA polynucleotide that can be transcribed into a sgRNA of sequences SEQ ID NO: 1 and SEQ ID NO: 3. In another more preferred embodiment, the inhibitor is selected from siRNA sequence SEQ ID NO: 10 and SEQ ID NO: 11.

The tumor that can be treated with the method of treatment of the invention is characterized by having increased copy number of the AC083973.1 gene, and/or decreased methylation of said gene and/or increased levels of the transcriptional product of said gene compared to a reference value.

The terms “reference value”, “AC083973.1 gene” and “transcriptional product” have been previously defined in connection with other aspects of the invention. All the particular and preferred embodiments of the other aspects of the invention regarding these terms fully apply to this aspect.

As used herein, the term “gene copy number” refers to the copy number of a nucleic acid molecule in a cell. The gene copy number includes the gene copy number in the genomic (chromosomal) DNA of a cell. In a normal cell (non-tumor cell), the gene copy number is normally two copies (one copy in each member of the chromosome pair). The gene copy number sometimes includes half of the gene copy number taken from samples of a cell population.

Methods to determine the copy number of the AC083973.1 are known in the art. Said methods include, without limitation, in situ hybridization (ISH) (such as fluorescence in situ hybridization (FISH), chromogenic in situ hybridization (CISH) or silver in situ hybridization (SISH)), genomic comparative hybridization or polymerase chain reaction (such as real time quantitative PCR). In an embodiment the copy number of the AC083973.1 gene is determined using qRT-PCR (real time quantitative PCR) from genomic DNA (gDNA). “Increased copy number” is understood as an increase in the copy number of the AC083973.1 gene in relation to the number of copies in a reference value. In a preferred embodiment, the tumor is characterized by having at least 1, at least 2, at least 3, at least 4, at least 5, at least 6, at least 7, at least 8, at least 9 or at least 10 or more copies of the AC083973.1 gene with respect to the reference value. In a more preferred embodiment, the tumor is characterized by having between 3 and 5 copies of the AC083973.1 gene. In a still more preferred embodiment, the tumor is characterized by having 4 copies of the AC083973.1 gene.

As used herein, the term “methylation” refers to the covalent attachment of a methyl group at the C5-position of the nucleotide base cytosine within the CpG dinucleotides of gene regulatory region. The term “methylation state” or “methylation status” refers to the presence or absence of 5-methyl-cytosine (“5-mCyt”) at one or a plurality of CpG dinucleotides within a DNA sequence. As used herein, the terms “methylation status” and “methylation state” are used interchangeably. A methylation site is a sequence of contiguous linked nucleotides that is recognized and methylated by a sequence-specific methylase. A methylase is an enzyme that methylates (i.e., covalently attaches a methyl group) one or more nucleotides at a methylation site.

The term “methylation status” refers to the presence or absence of 5-methylcytosine (“5-mCyt”) at one or a plurality of CpG dinucleotides present on the DNA sequence of a target DNA methylation gene. As used herein, the terms “methylation status” and “methylation state” are used interchangeably. Methylation status at one or more particular CpG methylation sites (each having two CpG dinucleotide sequences) within a DNA sequence include “unmethylated”, “fully-methylated” and “hemimethylated”.

The term “hypermethylation” or “increased methylation” refers to the average methylation state corresponding to an increased presence of 5-mCyt at one or a plurality of CpG dinucleotides within a DNA sequence of a test DNA sample, relative to a reference value.

The term “hypomethylation” or “decreased methylation” refers to the average methylation state corresponding to a decreased presence of 5-mCyt at one or a plurality of CpG dinucleotides within a DNA sequence of a test DNA sample, relative to a reference value. The methylation profile of a selected region in a DNA isolated from a sample may be determined by any assays known in the art. The genomic DNA is first isolated from the sample. Genomic DNA may be isolated by any means standard in the art, including the use of commercially available kits. Briefly, wherein the DNA of interest is encapsulated in by a cellular membrane the biological sample must be disrupted and lysed by enzymatic, chemical or mechanical means. The DNA solution may then be cleared of proteins and other contaminants e.g. by digestion with proteinase K. The genomic DNA is then recovered from the solution. This may be carried out by means of a variety of methods including salting out, organic extraction or binding of the DNA to a solid phase support. The choice of method will be affected by several factors including time, expense and required quantity of DNA. Wherein the sample DNA is not enclosed in a membrane (e.g. circulating DNA from a blood sample) methods standard in the art for the isolation and/or purification of DNA may be employed. Such methods include the use of a protein degenerating reagent e.g. chaotropic salt e.g. guanidine hydrochloride or urea; or a detergent e.g. sodium dodecyl sulphate (SDS), cyanogen bromide. Alternative methods include but are not limited to ethanol precipitation or propanol precipitation, vacuum concentration amongst others by means of a centrifuge. The person skilled in the art may also make use of devices such as filter devices e.g. ultrafiltration, silica surfaces or membranes, magnetic particles, polystyrol particles, polystyrol surfaces, positively charged surfaces, and positively charged membranes, charged membranes, charged surfaces, charged switch membranes, charged switched surfaces.

Once the nucleic acids have been extracted, the genomic double stranded DNA is used in the analysis, methylation analysis may be carried out by any means known in the art. A variety of methylation analysis procedures are known in the art and may be used to practice the invention. These assays allow for determination of the methylation state of one or a plurality of CpG sites within a tissue sample. In addition, these methods may be used for absolute or relative quantification of methylated nucleic acids. Such methylation assays involve, among other techniques, two major steps. The first step is a methylation specific reaction or separation, such as (i) bisulfite treatment, (ii) methylation specific binding, or (iii) methylation specific restriction enzymes. The second major step involves (i) amplification and detection, or (ii) direct detection, by a variety of methods such as (a) PCR (sequence-specific amplification) such as Taqman(R), (b) DNA sequencing of untreated and bisulfite-treated DNA, (c) sequencing by ligation of dye-modified probes (including cyclic ligation and cleavage), (d) pyrosequencing, (e) single-molecule sequencing, (f) mass spectroscopy, or (g) Southern blot analysis.

Additionally, restriction enzyme digestion of PCR products amplified from bisulfite-converted DNA may be used, e.g., the method described by Sadri and Hornsby (1996, Nucl. Acids Res. 24:5058-5059), or COBRA (Combined Bisulfite Restriction Analysis) (Xiong and Laird, 1997, Nucleic Acids Res. 25:2532-2534). COBRA analysis is a quantitative methylation assay useful for determining DNA methylation levels at specific gene loci in small amounts of genomic DNA. Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Frommer et al, 1992, Proc. Nat. Acad. Sci. USA, 89, 1827-1831). PCR amplification of the bisulfite converted DNA is then performed using primers specific for the CpG sites of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples. Typical reagents (e.g., as might be found in a typical COBRA-based kit) for COBRA analysis may include, but are not limited to: PCR primers for specific gene (or methylation-altered DNA sequence or CpG island); restriction enzyme and appropriate buffer; gene-hybridization oligo; control hybridization oligo; kinase labeling kit for oligo probe; and radioactive nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components.

In an embodiment the tumor is characterized by having decreased methylation of the AC083973.1 gene in relation to a reference value. In a preferred embodiment AC083973.1 has decreased methylation in the 5′ region of AC083973.1 gene. In a more preferred embodiment the decreased methylation is detected in a CpG selected from the group consisting of cg26394282 and cg16230352.

The term “cg26394282” refers to the position in chromosome 8, coordinates 42122810-42122811, according to hg19 (according to the release GRCh37 of February 2009).

The term “cg16230352” refers to the position in chromosome 8, coordinates 42123158-42123159, according to hg19 (according to the release GRCh37 of February 2009).

The level of the transcriptional product may be determined, without limitation, by any standard assays for determining mRNA expression levels such as qPCR, RT-PCR, RNA protection analysis, Northern blot, RNA dot blot, in situ hybridization, microarray technology, tag based methods such as serial analysis of gene expression (SAGE) including variants such as LongSAGE and SuperSAGE, microarrays, fluorescence in situ hybridization (FISH), including variants such as Flow-FISH, qFiSH and double fusion FISH (D-FISH), and the like.

In a preferred embodiment the transcriptional product of AC083973.1 is increased with respect to the reference value by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more. In another preferred embodiment the transcriptional product of AC083973.1 is any one, or any combination, of the variants ENST00000518213.1, ENST00000518994.1, ENST00000523459.5, ENST00000521802.5, ENST00000521470.1 and/or ENST00000520890.5. In a more preferred embodiment the transcriptional product is ENST00000521802.5 (SEQ ID NO: 16).

In a preferred embodiment, the inhibitor of functional expression of AC083973.1 is selected from sgRNA, a DNA polynucleotide that can be transcribed into a sgRNA, siRNA, shRNA, miRNA, an antisense oligonucleotide, and/or a ribozyme. In a more preferred embodiment, the inhibitor is selected from sgRNA and/or siRNA. In a more preferred embodiment, the inhibitor is selected from sgRNA sequence SEQ ID NO: 69 and SEQ ID NO: 70. In an even more preferred embodiment, the inhibitor comprises both sgRNA of sequences SEQ ID NO: 69 and SEQ ID NO: 70. In another more preferred embodiment, the inhibitor is selected from a DNA polynucleotide that can be transcribed into a sgRNA selected from SEQ ID NO: 1 and SEQ ID NO: 3. In another even more preferred embodiment, the inhibitor comprises both DNA polynucleotides that can be transcribed into a sgRNA of sequences SEQ ID NO: 1 and SEQ ID NO: 3. In another more preferred embodiment, the inhibitor is selected from siRNA sequence SEQ ID NO: 10 and SEQ ID NO: 11.

All the terms and embodiments previously described are equally applicable to this aspect of the invention.

### Method of Diagnosing and Treating a Tumor

In another aspect the invention relates to a method of diagnosing and treating a tumor in a subject comprising:

(i) determining the copy number of the AC083973.1 gene, and/or the methylation level of said gene and/or the level of the transcriptional product of said gene in a sample,
(ii) comparing the copy number and/or methylation level and/or level obtained under (i) with a reference value,
(iii) diagnosing the subject as having a tumor susceptible of being treated by an inhibitor according to the invention when the copy number of the gene is increased and/or the methylation level is decreased and/or the level of the transcriptional product of the gene is increased compared to the reference value and
(iv) administering the subject diagnosed with the tumor with a therapeutic effective amount of an inhibitor of functional expression of AC083973.1 according to the invention.

The terms “treating”, “tumor”, “subject”, “copy number”, “AC083973.1 gene”, “methylation level”, “transcriptional product”, “reference value” and “therapeutic effective amount” have been previously defined in connection with other aspects of the invention. All the particular and preferred embodiments of the other aspects of the invention regarding these terms fully apply to this aspect.

The term “diagnosing” as disclosed herein means the process of determining which disease or condition explains a person's symptoms and signs.

In a particular embodiment, the method for diagnosing and treating a tumor of the invention comprises determining the copy number of the AC083973.1 gene. Methods to determine the copy number of the AC083973.1 gene have been disclosed previously. In an embodiment the copy number of the AC083973.1 gene is determined using qRT-PCR from genomic DNA (gDNA). In a preferred embodiment, when the copy number of the AC083973.1 gene is increased with respect to a reference value, the subject is diagnosed as having a tumor susceptible of being treated by an inhibitor according to the invention and a therapeutic effective amount of the inhibitor of the invention is administered to the subject. The term “increased copy number” has been previously defined. In a more particular embodiment, when the copy number of the AC083973.1 gene is at least 1, at least 2, at least 3, at least 4, at least 5, at least 6, at least 7, at least 8, at least 9, at least 10 or more copies of the AC083973.1 gene with respect to a reference value, the subject is diagnosed as having a tumor susceptible of being treated by an inhibitor according to the invention and administered a therapeutic effective amount of the inhibitor of the invention. In a more preferred embodiment if the copy number of the AC083973.1 gene is between 3 and 5 copies, the subject is diagnosed as having a tumor susceptible of being treated by an inhibitor according to the invention and administered a therapeutic effective amount of the inhibitor of the invention. In a still more preferred embodiment, if the copy number of the AC083973.1 gene is 4, the subject is diagnosed as having a tumor susceptible of being treated by an inhibitor according to the invention and administered a therapeutic effective amount of the inhibitor of the invention.

In a particular embodiment, the therapeutic effective amount of the inhibitor according to the invention, preferably an siRNA, is an amount that when administered to the subject an intracellular concentration of from about 1 nanomolar (nM) to about 100 nM, preferably from about 2 nM to about 50 nM, more preferably from about 20 nM to about 40 nM is achieved in tumor cells In a more particular embodiment the therapeutic effective amount of the inhibitor according to the invention, preferably an siRNA, is an amount that when administered to a subject an intracellular concentration of 30 nM is achieved in the tumor cells. It is contemplated that greater or lesser amounts of the inhibitor can be administered.

In a particular embodiment, the method for diagnosing and treating a tumor susceptible of being treated by an inhibitor according to the invention comprises determining the methylation level of the AC083973.1 gene. Methods to determine the methylation level of the AC083973.1 gene have been disclosed previously. In an embodiment, when the methylation level of the AC083973.1 gene is decreased with respect to a reference value, the subject is diagnosed as having a tumor and a therapeutic effective amount of the inhibitor of the invention is administered to the subject. In an embodiment AC083973.1 has decreased methylation in relation to a reference value. In a particular embodiment, when the methylation level of the AC083973.1 gene in the 5′ region is decreased in relation to a reference value, the subject is diagnosed as having a tumor and administered a therapeutic effective amount of the inhibitor of the invention. In a more particular embodiment, the decreased methylation is detected in a CpG selected from the group consisting of cg26394282 and cg16230352.

In a particular embodiment, the method for diagnosing and treating a tumor susceptible of being treated by an inhibitor according to the invention comprises determining the level of the transcriptional product of the AC083973.1 gene. Methods to determine the level of the transcriptional product of the AC083973.1 gene have been disclosed previously. In an embodiment, when the level of the transcriptional product of AC083973.1 is increased with respect to the reference value by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more, the subject is diagnosed as having a tumor and a therapeutic effective amount of the inhibitor of the invention is administered to the subject. In another preferred embodiment the transcriptional product of AC083973.1 is any one, or any combination, of the variants ENST00000518213.1, ENST00000518994.1, ENST00000523459.5, ENST00000521802.5, ENST00000521470.1 and/or ENST00000520890.5. In a more preferred embodiment the transcriptional product is ENST00000521802.5 (SEQ ID NO: 16).

In a preferred embodiment the subject is diagnosed as having a tumor susceptible of being treated by an inhibitor according to the invention when the copy number of the AC083973.1 gene is increased with respect to a reference value and/or when the AC083973.1 gene has decreased methylation in relation to a reference value and/or when the transcriptional product of AC083973.1 is increased with respect to the reference value.

The term “sample” as used in the present invention, it refers to biological material isolated from a subject. The sample contains any material suitable for detecting the level of expression of a gene and may be a material comprising genetic material of the subject. The biological sample may comprise cellular and/or non-cellular material of the subject, preferably cellular material. In a particular embodiment, the sample comprises genetic material, for example, DNA, genomic DNA (gDNA), complementary DNA (cDNA), RNA, mRNA, etc., of the subject under study. In a particular embodiment, the genetic material is RNA. The sample can be isolated from any biological tissue or fluid, such as, for example, blood, saliva, plasma, serum, urine, cerebrospinal fluid (CSF), feces, nasal, buccal or bucco-pharyngeal swabs, a specimen, a specimen obtained from a biopsy, and a sample of tissue embedded in paraffin. The procedures for isolating samples are well known to those skilled in the art.

Before analyzing the sample, it will often be desirable to perform one or more operations of preparing said sample to separate the molecule to be determined from other molecules that are in the sample. In a particular embodiment, the molecules are nucleic acids, DNA and/or RNA. These sample preparation operations include manipulations such as: concentration, suspension, extraction of intracellular material (e.g., nucleic acids from tissue samples/whole cell and the like), nucleic acid amplification, fragmentation, transcription, labeling and/or extension reactions. These methods are well known to a person skilled in the art. Commercial kits for mRNA purification are also available, including, without limitation, miRNeasy Mini Kit from Qiagen, miRNA Life Technologies isolation kits, mirPremier the microRNA isolation kit from Sigma-Aldrich and High Pure miRNA Isolation Kit from Roche. In a particular embodiment the integrity of the RNA was analyzed using RNA 6000 Nano Chips in the Agilent 2100 bionalizer (Agilent Technologies, Palo Alto, Calif., USA). In a particular embodiment, the sample is a tumor sample.

In an embodiment if the subject is diagnosed as having a tumor susceptible of being treated by an inhibitor according to the invention, then it is administered with a therapeutic effective amount of an inhibitor of functional expression of AC083973.1 according to the invention. In a preferred embodiment, the therapeutic effective amount reduces the levels of any of the AC083973.1 gene products by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% with respect to a reference value. In more preferred embodiment the therapeutic effective amount reduces the levels of any one, or any combination, of the variants selected from: ENST00000518213.1, ENST00000518994.1, ENST00000523459.5, ENST00000521802.5, ENST00000521470.1 and/or ENST00000520890.5. In a still more preferred embodiment the therapeutic effective amount reduces the levels of ENST00000521802.5 (SEQ ID NO: 16) expression.

In a particular embodiment, the therapeutic effective amount of the inhibitor according to the invention, preferably an siRNA, is an amount that when administered to the subject an intracellular concentration of from about 1 nanomolar (nM) to about 100 nM, preferably from about 2 nM to about 50 nM, more preferably from about 20 nM to about 40 nM is achieved in tumor cells In a more particular embodiment the therapeutic effective amount of the inhibitor according to the invention, preferably an siRNA, is an amount that when administered to a subject an intracellular concentration of 30 nM is achieved in the tumor cells. It is contemplated that greater or lesser amounts of the inhibitor can be administered.

In a preferred embodiment the inhibitor is selected from a sgRNA, a DNA polynucleotide that can be transcribed into a sgRNA or a siRNA. In a more preferred embodiment the CRISPR-sgRNA is selected from SEQ ID NO: 69 and SEQ ID NO: 70, the DNA polynucleotide that can be transcribed into a sgRNA is selected from SEQ ID NO: 1 and SEQ ID NO: 3 and/or the siRNA is selected from SEQ ID NO: 10 or SEQ ID NO: 11. In an even more preferred embodiment, the inhibitor comprises both sgRNA of sequences SEQ ID NO: 69 and SEQ ID NO: 70 or both DNA polynucleotides that can be transcribed into a sgRNA of sequences SEQ ID NO: 1 and SEQ ID NO: 3.

In a preferred embodiment the tumor is lung cancer and/or head and neck cancer. In a more preferred embodiment the lung cancer is non-small cell lung cancer. In a more preferred embodiment the non-small cell lung cancer is squamous cell carcinoma or adenocarcinoma. In another more preferred embodiment the head and neck cancer is head and neck squamous cell carcinoma.

All the terms and embodiments previously described are equally applicable to this aspect of the invention.

Method for Selecting a Therapy for a Patient with a Tumor

In another aspect, the invention relates to a method for selecting a therapy for treating a subject with a tumor comprising:

(i) determining the copy number of the AC083973.1 gene, and/or the methylation level of said gene and/or the level of the transcriptional product of said gene in a sample,
(ii) comparing the copy number and/or methylation level and/or level obtained under (i) with a reference value,
(iii) selecting a therapy consisting of an inhibitor of functional expression of AC083973.1 according to claim 1 if the copy number of the gene is increased and/or the methylation level is decreased and/or the level of the transcriptional product of the gene is increased compared to the reference value.

The terms “tumor”, “subject”, “copy number”, “AC083973.1 gene”, “methylation level”, “transcriptional product”, “sample” and “reference value” and “therapeutic effective amount” have been previously defined in connection with other aspects of the invention. All the particular and preferred embodiments of the other aspects of the invention regarding these terms fully apply to this aspect.

In an embodiment the copy number of the AC083973.1 gene is determined using qRT-PCR from genomic DNA (gDNA). In a preferred embodiment, when the copy number of the AC083973.1 gene is increased with respect to a reference value, a therapy consisting of an inhibitor according to the invention is selected for treating the subject. The term “increased copy number” has been previously defined. In a more particular embodiment, when the copy number of the AC083973.1 gene is at least 1, at least 2, at least 3, at least 4, at least 5, at least 6, at least 7, at least 8, at least 9, at least 10 or more copies of the AC083973.1 gene with respect to a reference value, a therapy consisting of an inhibitor according to the invention is selected for treating the subject. In a more preferred embodiment if the copy number of the AC083973.1 gene is between 3 and 5 copies, a therapy consisting of an inhibitor according to the invention is selected for treating the subject. In a still more preferred embodiment, if the copy number of the AC083973.1 gene is 4, a therapy consisting of an inhibitor according to the invention is selected for treating the subject.

In a particular embodiment, the method for selecting a therapy of the invention comprises determining the methylation level of the AC083973.1 gene. Methods to determine the methylation level of the AC083973.1 gene have been disclosed previously. In an embodiment, when the methylation level of the AC083973.1 gene is decreased with respect to a reference value, a therapy consisting of an inhibitor according to the invention is selected for treating the subject. In an embodiment AC083973.1 has decreased methylation in relation to a reference value. In a particular embodiment, when the methylation level of the AC083973.1 gene in the 5′ region is decreased in relation to a reference value, a therapy consisting of an inhibitor according to the invention is selected for treating the subject. In a more particular embodiment, the decreased methylation is detected in a CpG selected from the group consisting of cg26394282 and cg16230352.

In a particular embodiment, the method for selecting a therapy of the invention comprises determining the level of the transcriptional product of the AC083973.1 gene. Methods to determine the level of the transcriptional product of the AC083973.1 gene have been disclosed previously. In an embodiment, when the level of the transcriptional product of AC083973.1 is increased with respect to the reference value by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, or more, a therapy consisting of an inhibitor according to the invention is selected for treating the subject. In another preferred embodiment the transcriptional product of AC083973.1 is any one, or any combination, of the variants ENST00000518213.1, ENST00000518994.1, ENST00000523459.5, ENST00000521802.5, ENST00000521470.1 and/or ENST00000520890.5. In a more preferred embodiment the transcriptional product is ENST00000521802.5 (SEQ ID NO: 16).

In a preferred embodiment, a therapy consisting of an inhibitor according to the invention is selected for treating the subject when the copy number of the AC083973.1 gene is increased with respect to a reference value and/or when the AC083973.1 gene has decreased methylation in relation to a reference value and/or when the transcriptional product of AC083973.1 is increased with respect to the reference value.

In a preferred embodiment the inhibitor is selected from a sgRNA, a DNA polynucleotide that can be transcribed into a sgRNA or a siRNA. In a more preferred embodiment the CRISPR-sgRNA is selected from SEQ ID NO: 69 and SEQ ID 70, the DNA polynucleotide that can be transcribed into a sgRNA selected from SEQ ID NO: 1 and SEQ ID NO: 3 and/or the siRNA is selected from SEQ ID NO: 10 or SEQ ID NO: 11. In an even more preferred embodiment, the inhibitor comprises both sgRNA of sequences SEQ ID NO: 69 and SEQ ID NO: 70 or both DNA polynucleotides that can be transcribed into a sgRNA of sequences SEQ ID NO: 1 and SEQ ID NO: 3.

In a preferred embodiment the tumor is lung cancer and/or head and neck cancer. In a more preferred embodiment the lung cancer is non-small cell lung cancer. In a more preferred embodiment the non-small cell lung cancer is squamous cell carcinoma or adenocarcinoma. In another more preferred embodiment the head and neck cancer is head and neck squamous cell carcinoma.

All the terms and embodiments previously described are equally applicable to this aspect of the invention.

### Method for Increasing the Migration of Cytotoxic Immune Cells

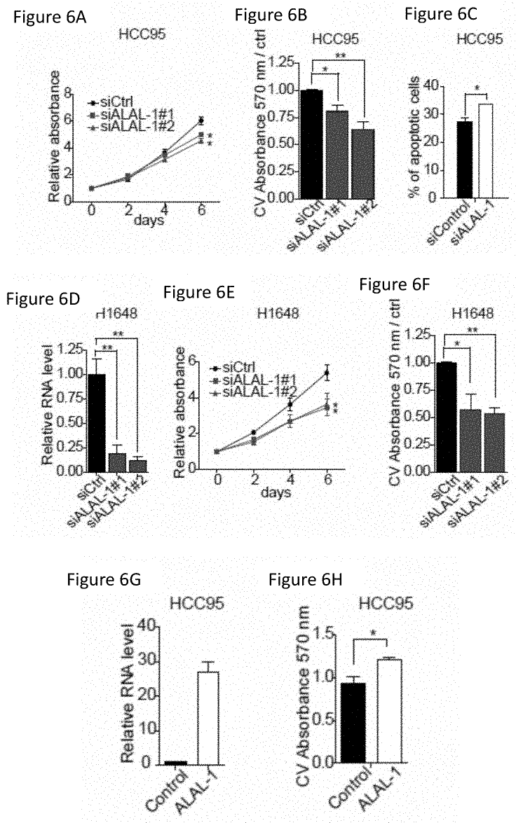

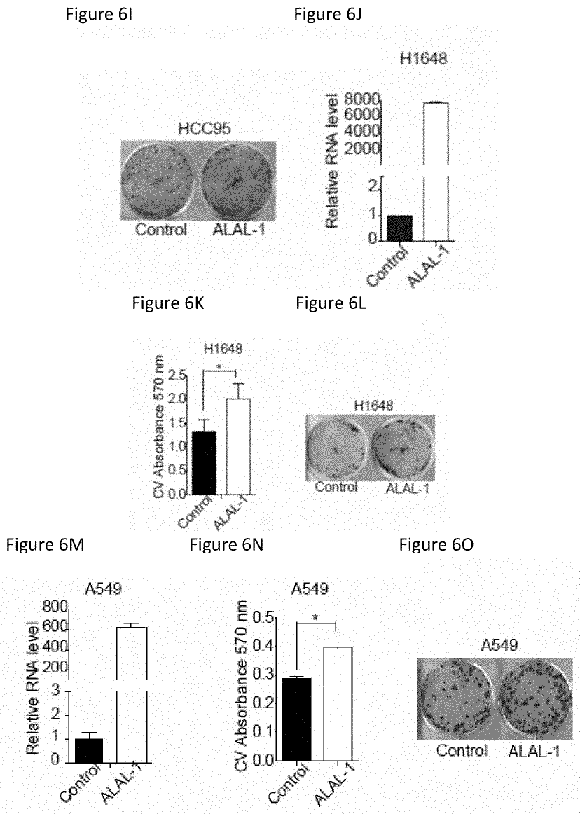

The inventors have found that the inhibition of the functional expression of AC083973.1 gene with siRNAs increased migration of CD8+ T lymphocytes, CD19+ lymphocytes, NK cells and monocytes towards the tumor cells (FIG. 6C).

Therefore, in another aspect the invention relates to a method for increasing the migration of cytotoxic immune cells towards a tumor comprising administering the inhibitor according to the invention to a subject suffering from said tumor, wherein the tumor has increased copy number of the AC083973.1 gene and/or decreased methylation of said gene and/or increased level of the transcriptional product of said gene compared to a reference value.

The terms “tumor”, “subject”, “copy number”, “AC083973.1 gene”, “methylation”, “transcriptional product” and “reference value” have been previously defined in connection with other aspects of the invention. All the particular and preferred embodiments of the other aspects of the invention regarding these terms fully apply to this aspect.

As disclosed herein “immune cell” refers to a cell of the immune system, which originates in the bone, matures and migrates to guard the peripheral tissues, circulating in blood and lymph vessels. Immune cells originate from a pluripotent haematopoietic stem cell, which gives rise to lymphoid lineages responsible for adaptive immunity, and also to myeloid lineages that participate in both innate and adaptive immunity. The lymphoid lineages includes cells that differentiate into natural killer cells (NK cells), T cells and B cells, and the myeloid lineages include cells that differentiate into monocytes and macrophages, dendritic cells, neutrophils, basophils, and eosinophils.

The term “cytotoxic immune cell” as disclosed herein means a T cell that usually bears CD8 molecular markers on its surface and that functions in cell-mediated immunity by destroying a cell having a specific antigenic molecule on its surface.

In a particular embodiment, the method increases the migration of a cytotoxic immune cell selected from the group consisting of a T lymphocyte, more particularly a CD8⁺ T lymphocyte, a B lymphocyte, more particularly a CD19⁺ lymphocyte, a NK cell or a monocyte.

The term “natural killer cell” or “NK cell”, as used herein, refers to a type of cytotoxic lymphocyte that provides rapid responses to viral-infected cells and responds to tumor formation. NK cells are characterized for expressing CD16 and CD56.

The term “T lymphocyte” or “T cell”, as used herein, refers to a type of lymphocyte characterized by expressing a T-cell receptor (TCR) on the cell surface, which plays a central role in cell-mediated immunity. There are several types of T cells, including helper T cells (CD4+), cytotoxic T cells (CD8+), memory T cells (CD45RO), regulatory T cells (Tregs) (CD4+CD25brightFoxp3+ or induced CD4+CD25bright cells), and natural killer T cells (NKT cells). Upon activation of the T cells, they begin expressing the so-called “early and/or intermediate activation markers”. “Early and/or intermediate T cell-activation markers” include CD69, HLA-DR, CD25, CD71, CD154, CD38, and CD27. In a particular embodiment, the T lymphocyte is a CD8⁺ lymphocyte.

The term “B lymphocyte” or “B cell”, as used herein, refers to a lymphocyte that plays a role in humoral immunity of the adaptive immune system, and which is characterized by the presence of the B cell receptor (BCR) on the cell surface. B cell types include plasma cells, memory B cells, B-1 cells, B-2 cells, marginal-zone B cells, follicular B cells, and regulatory B cells (Breg). In a particular embodiment, the B lymphocyte is a CD19⁺ lymphocyte.

The term “monocyte”, as used herein, refers to an immune cell that circulates in the blood for about one to three days and then migrates from the bloodstream to other tissues where it will then differentiate into tissue resident macrophages or dendritic cells.

The migration response of the cytotoxic immune cells towards a tumor can be measured by any method known in the art. Such methods include, without being limited to, any cell detection technique, such as those based on antigen-antibody interaction. Non-liming examples include cytometry and immunohistochemistry. Non-limiting examples also include any cell migration assay, such as a trans-well assay. As disclosed herein a “trans-well assay” is a commonly used test to study the migratory response of cells to inducers or inhibitors. During this assay, the cells are placed on the upper layer of a cell culture insert with permeable membrane and a solution containing the test agent is placed below the cell permeable membrane. Following an incubation period, the cells that have migrated through the membrane are stained and counted. The membrane may be coated with some extracellular matrix component (e.g. collagen) which facilitates both adherence and migration.

In a preferred embodiment the migration response of the cytotoxic immune cells towards a tumor is measured by a trans-well assay.

As disclosed herein the migration response of the cytotoxic immune cells towards a tumor can be increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% with respect to a reference value. In a preferred embodiment the migration response of the cytotoxic immune cells towards a tumor is in increased between 50 and 75% with respect to a reference value.

In a particular embodiment, the therapeutic effective amount of the inhibitor according to the invention, preferably an siRNA, is an amount that when administered to the subject an intracellular concentration of from about 1 nanomolar (nM) to about 100 nM, preferably from about 2 nM to about 50 nM, more preferably from about 20 nM to about 40 nM is achieved in tumor cells In a more particular embodiment the therapeutic effective amount of the inhibitor according to the invention, preferably an siRNA, is an amount that when administered to a subject an intracellular concentration of 30 nM is achieved in the tumor cells. It is contemplated that greater or lesser amounts of the inhibitor can be administered.

In a preferred embodiment, the inhibitor is selected from sgRNA, a DNA polynucleotide that can be transcribed into a sgRNA, siRNA, shRNA, miRNA, an antisense oligonucleotide, and/or a ribozyme. In a more preferred embodiment, the inhibitor is selected from sgRNA and/or siRNA. In a more preferred embodiment, the inhibitor is selected from sgRNA sequence SEQ ID NO: 69 and SEQ ID NO: 70. In an even more preferred embodiment, the inhibitor comprised both sgRNA sequences SEQ ID NO: 69 and SEQ ID NO: 70. In another more preferred embodiment, the inhibitor is selected from the DNA polynucleotide that can be transcribed into a sgRNA selected from SEQ ID NO: 1 and SEQ ID NO: 3. In another more preferred embodiment, the inhibitor comprises both DNA polynucleotides that can be transcribed into a sgRNA of sequences SEQ ID NO: 1 and SEQ ID NO: 3. In another more preferred embodiment, the inhibitor is selected from siRNA sequence SEQ ID NO: 10 and SEQ ID NO: 11.

In a preferred embodiment, the inhibitor is administered when the copy number of the AC083973.1 gene is increased by at least 1, at least 2, at least 3, at least 4, at least 5, at least 6, at least 7, at least 8, at least 9 or at least 10 more copies of the AC083973.1 gene in relation to a reference value. In a more preferred embodiment, the inhibitor is administered when the AC083973.1 gene copy number is 4.

In another preferred embodiment, the inhibitor is administered when the tumor has decreased methylation of the AC083973.1 gene in relation to a reference value. In a preferred embodiment, AC083973.1 has decreased methylation in the 5′ region. In a more preferred embodiment the decreased methylation is detected in a CpG selected from the group consisting of cg26394282 and cg16230352.

In another preferred embodiment, the inhibitor is administered when the tumor has increased levels of any one of the transcriptional products of the AC083973.1 gene or genes compared to a reference value. In a more preferred embodiment the inhibitor is administered when the tumor has increased level of any one, or any combination of, the variants selected from: ENST00000518213.1, ENST00000518994.1, ENST00000523459.5, ENST00000521802.5, ENST00000521470.1 and/or ENST00000520890.5. In a still more preferred embodiment the inhibitor is administered when the tumor has increased levels of the transcriptional product ENST00000521802.5 (SEQ ID NO: 16).

All the terms and embodiments previously described are equally applicable to this aspect of the invention.

The invention is described herein by way of the following examples which are merely illustrative and not limitative of the scope of the invention.

### Examples

### Materials and Methods

### Patients

Copy number tumor data were retrieved from several previously published datasets, including TCGA data for 7,448 tumors across 25 cancer types downloaded from the Firebrowser server (2013_10), 85 lung adenocarcinoma tumors from CIMA-CUN GSE72195 (Aramburu et al., 2015. BMC Genomics 16, 752), 162 from The University of Texas MD Anderson (MDA) Cancer Center GSE72195 (Aramburu et al., 2015), as well as 101 from Uppsala University GSE28582 (Micke et al., 2011. J Thorac Oncol 6, 1833-1840). The validation set of LUSC patients was obtained from the CIBERES multi-institutional Pulmonary Biobank Platform (Spain).

### Data Processing

The TCGA downloaded data contains the results of copy number alterations obtained from the analysis of Affymetrix 6.0 SNP arrays using GISTIC algorithm. Using in house R and perl scripts, copy number alterations were annotated (GENCODEv19) and classified (biotypes). The expression data was retrieved from MiTranscriptome and TCGA. From additional lung cancer cohorts (GSE18842, GSE19804, GSE19188) expression of ALAL-1 was obtained with the probe 231378_at. Methylation data (HumanMethylation 450K BeadChip was retrieved from TCGA Wanderer interface (Diez-Villanueva et al., 2015. Epigenetics Chromatin 8, 22). In addition CCLE resources (http://www.broadinstitute.org/ccle) were used to assess ALAL-1 expression in cancer cell lines.

### Pathway Enrichment Analysis

Enrichment analysis of Gene Ontologies (GO) from the differentially expressed genes were obtained with the R package clusterProfiler (Yu et al., 2012. OMICS 16, 284-287). The Ingenuity Pathway Analysis (IGA) was used for additional data interpretation.

### CRISPR/Cas9 Editing

CRISPR/Cas9 single guide RNAs (sgRNAs) were designed with the tool available at http://crispr.mit.edu/. To delete exon D of ALAL-1 the two sgRNAs with the highest score were cloned in pX330 as described in the Zhang lab CRISPR protocol (Ran F A et al., 2013. Nat. Protoc. 8(11): 2281-2308; doi: 10.1038/nprot.2013.143) (https://www.addgene.org/crispr/zhang/). The sequences of the sgRNAs used are listed below. To generate the genomic deletion, HCC95 cells were cotransfected with pX330-sgRNA-1 and pX330-sgRNA-2 and GFP expressing plasmid. Control cells were transfected with pX330 vector lacking any sgRNA. 24 hours post-transfection, GFP positive cells were sorted in six 96 well-plates using the BD FACSAria IIu cytometer. Cells were left to grow until they reached confluency. Genomic DNA was then extracted with the QuickExtract reagent (Epicentre). Genotyping was performed with PCR primers upstream and downstream of the sgRNA cleavage sites. PCR products were then run in an agarose gel to check for amplicon size. PCR products of the clones carrying the deletion were sent for Sanger sequencing.

TABLE 2
CRISPR-Cas9 sgRNAs DNA polynucleotides

| | | SEQ |
|---|---|---|
| CRISPR-Cas9 sgRNAs | | ID |
| Target gene | Sequence.sgRNAs | NO |
| ALAL1_LEFT.exonD_sense | CACCGTTCCCAGATGGGGCGACGGC | 1 |
| ALAL1_LEFT.exonD_antisense | AAACGCCGTCGCCCCATCTGGGAAC | 2 |
| ALAL1_RIGHT.exonD_sense | CACCGTATATTGCTAAGACGTTGAC | 3 |
| ALAL1_RIGHT.exonD_antisense | AAACGTCAACGTCTTAGCAATATAC | 4 |
| sgRNA-1_LEFT | CACCGUUCCCAGAUGGGGCGACGGC | 69 |
| sgRNA-2_RIGHT | CACCGUAUAUUGCUAAGACGUUGAC | 70 |

TABLE 3
qPCR primers

| | | SEQ |
|---|---|---|
| Target | | ID |
| gene.genomic.DNA | qPCR.primer.genotyping | NO |
| ALAL1_fwd_in | TTGATGTCCCCAATATACCTGA | 5 |
| ALAL1_rev_in | TGTGGTTTCTTTATGTGGTTGC | 6 |
| ALAL1_fwd_out | TGCCCACTGTCTGGCAAA | 7 |
| ALAL1_rev_out | CCACCATGCTTGGCACTAT | 8 |

### Clonogenicity, Cell Proliferation and Apoptosis Assays

Approximately 500 cells were plated in each well of a 6-well plate. After a period of 10-14 days, cells were fixed using 0.5% glutaraldehyde and stained with 1% crystal violet in 35% methanol. Counting colonies was performed manually. Crystal violet staining was solubilized with 10% acetic acid and absorbance was measured at 570 nm on a spectrophotomer plate reader. For the cell proliferation assays, 1×103 HCC95, 1.5×103 H1648 or 500 A549 cells were plated in each well of a 96-well plate, and its proliferation was assessed using the CellTiter Aqueous Non-Radioactive Cell Proliferation Assay kit (MTS) (Promega). Apoptosis was measured by flow cytometry using the Annexin V and 7-AAD staining kit (BD Biosciences) and the FlowJO analysis software.

### Cell Culture, RNAi and TNF-α Treatment

All the cell lines used were cultured at 37° C. in the presence of 5% CO₂ using either DMEM or RPMI 1640 medium (GIBCO) supplemented with 10% fetal bovine serum and 1% of PenStrep. For ALAL-1 inhibition, 2×10⁵ cells per well were plated in a 6-well plate. The next day siRNAs at a final concentration of 30 nM siRNA were transfected using Lipofectamine 2000 (Invitrogen) following manufacturers protocol. siRNAs are listed below. The TNF-α treatment was performed using a concentration of 10 ng/ml of TNF-α (R&D systems, 210-TA) for the indicated time points.

TABLE 4
siRNA sequences

| siRNA sequences | | SEQ |
|---|---|---|
| siRNAs | Sequence | ID NO |
| CTRL_siRNA | CAGUCGCGUUUGCGACUGGC | 9 |
| ALAL-1_siRNA-1 | GGUAUAUUGGGGACAUCAA | 10 |
| ALAL-1_siRNA-2 | GGAUAUGGAGAAAAUUAUU | 11 |
| p65_siRNA | GCCCUAUCCCUUUACGUCA | 12 |

### RNA-seq

Total RNA from HCC95 cells was extracted using Maxwell® 16 Total RNA Purification Kit (Promega). Triplicates for each condition (ALAL-1 inhibition, TNF-α treatment and controls) were done. RNA quality for each sample was assessed using Agilent 2200 TapeStation. Library preparation was performed with the TruSeq Stranded mRNA kit (Illumina). Libraries were then sequenced on an Illumina NextSeq (75 bp paired-end). Sequenced reads were aligned using bowtie2 (against hg19) and the differential gene expression analysis was carried out with DeSeq2. The RNA-seq raw is available at the GEO under the accession code GSE114632.

### DNA Extraction and Gene Copy Number Estimation

Genomic DNA was obtained from 2×105 cells using DNA extraction kit (QIAGEN). Copy number was assessed by qPCR using primers recognizing ALAL-1 locus. Data was normalized to PEX19 gene located in chromosome 1p36.23, a region with no significant aneuploidy in the cancer cell lines studied. Copy number of ALAL-1 locus in the CRISPR/Cas9 engineered cells was quantified using the same methodology.

RNA Extraction, qPCR, and Primer Design

Total RNA was extracted using Trizol reagent (Sigma). After DNAseI (Invitrogen) treatment RNA was reverse transcribed with the High Capacity Kit (Applied Biosystems). RT-PCRs was performed in quatriplicates and relative gene expression was obtained using HPRT as the endogenous control for data normalization. All primers were designed using the Universal ProbeLibrary Assay Design Center (Roche).

TABLE 5
qPCR primers

| qPCR primers | | | |
|---|---|---|---|
| Target.gene | sequence | use | SEQ ID NO |
| ALAL1_gDNA_fwd | TTGATGTCCCCAATATACCTGA | gDNA | 19 |
| ALAL1_gDNA_rev | TGTGGTTTCTTTATGTGGTTGC | gDNA | 20 |
| PEX19_gDNA_fwd | AGCCAGCCAGGGCTTACTC | gDNA | 21 |
| PEX19_gDNA_rev | CTGAGGTCAACCTGCTCACA | gDNA | 22 |
| ALAL1_fwd_IS | GACAGAATCTGGCAACAGATGA | qPCR | 23 |
| ALAL1_rev_IS | TTGATGTCCCCAATATACCTGA | qPCR | 24 |
| ALAL1_fwd_exon2 | AGCCGGAAACACAGAAAGG | qPCR | 25 |
| ALAL1_rev_exon2 | GGGATCCCAATTGTAGAGCA | qPCR | 26 |
| ALAL1_fwd_exon3 | AAAGGTCACTTCCTGGCAGA | qPCR | 27 |
| ALAL1_rev_exon3 | GGAATCTCAGGTTAGACTTTCCA | qPCR | 28 |
| IKBKB_fwd | AATGGAGCAGGCTGTGGA | qPCR | 29 |
| IKBKB_rev | CATGGGGCTCCTCTGTAAGT | qPCR | 30 |
| PLAT_fwd | GCTGACGTGGGAGTACTGTG | qPCR | 31 |
| PLAT_rev | CTGAGGCTGGCTGTACTGTCT | qPCR | 32 |
| HPRT_fwd | TGACACTGGCAAAACAATGCA | qPCR | 33 |
| HPRT_rev | GGTCCTTTTCACCAGCAAGCT | qPCR | 34 |
| MALAT1_fwd | GACGGAGGTTGAGATGAAGC | qPCR | 35 |
| MALAT1_rev | ATTCGGGGCTCTGTAGTCCT | qPCR | 36 |
| U6_fwd | GCTTCGGCAGCACATATACTAAAAT | qPCR | 37 |
| U6_rev | ACGAATTTGCGTGTCATCCTT | qPCR | 38 |
| p65_fwd | TCATGAAGAAGAGTCCTTTCAGC | qPCR | 39 |
| p65_rev | GGATGACGTAAAGGGATAGGG | qPCR | 40 |
| SART3_fwd | AGAAGGGTTGATTTCAAACAAGA | qPCR | 41 |
| SART3_rev | CTCCAAGGCACGAGTAAAGG | qPCR | 42 |
| IL8_fwd | GAGTGGACCACACTGCGCCA | qPCR | 43 |
| IL8_rev | TCCACAACCCTCTGCACCCAGT | qPCR | 44 |
| BCL3_fwd | ACAACAACCTACGGCAGACA | qPCR | 45 |
| BCL3_rev | CCACAGACGGTAATGTGGTG | qPCR | 46 |
| MEFV_fwd | GCCAGGACTCCCATGAAA | qPCR | 47 |
| MEFV_rev | GCTTCAGGTGGCGCTTAC | qPCR | 48 |
| TNFAIP6_fwd | GGCCATCTCGCAACTTACA | qPCR | 49 |
| TNFAIP6_rev | GCAGCACAGACATGAAATCC | qPCR | 50 |
| CXCL10_fwd | TTCTGATTTGCTGCCTTATC | qPCR | 51 |
| CXCL10_rev | CTTGGATTAACAGGTTGATTACT | qPCR | 52 |
| IL1alpha_fwd | GGTTGAGTTTAAGCCAATCCA | qPCR | 53 |
| IL1alpha_rev | TGCTGACCTAGGCTTGATGA | qPCR | 54 |
| IL36G_fwd | AGAGGCACTCCAGGAGACG | qPCR | 55 |
| IL36G_rev | CTGAAGGGTCCACACTTGCT | qPCR | 56 |
| TNIP3_fwd | AAGGACGACAGGCAGAGAGA | qPCR | 57 |
| TNIP3_rev | TTCTTCATTTAGGCGTTCCTTT | qPCR | 58 |
| IL6_fwd | GCCTTCGGTCCAGTTGCCTT | qPCR | 59 |
| IL6_rev | AGTGCCTCTTTGCTGCTTTCA | qPCR | 60 |
| SAA2_fwd | AGGCTCAGACAAATACTTCCATGC | qPCR | 61 |
| SAA2_rev | TCTCTGGCATCGCTGATCACTTCT | qPCR | 62 |
| TRIM31_fwd | AACCTGTCACCATCGACTGTG | qPCR | 63 |
| TRIM31_rev | TGATTGCGTTCTTCCTTACGG | qPCR | 64 |
| EDN2_fwd | TCCTGGCTCGACAAGGAGT | qPCR | 65 |
| EDN2_rev | GGTTTCCCAGGCCGTAAG | qPCR | 66 |

### RNA Pulldown and RIP

RNA pulldown was performed as previously described (Marín-Béjar et al., 2013. Genome Biol. 14, R104) using a total protein extract of HCC95 cells. For mass spectrometry analysis the differential bands were submitted to Taplin Mass Spectrometry Facility (Harvard). RIP experiment 1×107 A549 cells overexpressing ALAL-1 were lysed with lysis buffer (20 mM Tris-HCl at pH 7.5, 100 mM KCl, 5 Mm MgCl₂, 0.5% NP-40, protease inhibitors (Roche), RNase inhibitor (100 U/ml) and 10 mM DTT). Protein lysate was then incubated with pre-washed Protein-A magnetic beads for 1 hour with rotation at 4° C., for pre-clearing. Extract was diluted up to 1 ml with RIP Buffer and incubated either with Normal Rabbit IgG (Cell Signaling, 2729S) or with anti-SART3 (abcam, ab155765), overnight with rotation at 4° C. Protein-A magnetic beads were added for one hour, and then washed five times with Buffer A (150 mM KCl, 25 mM Tris pH 7.4, 5 mM EDTA, 0.5% NP-40, 0.5 mM DTT), for the last wash PBS was used. RNA was recovered from the beads by resuspending them with Trizol Reagent.

### ALAL-1 and SART3 Overexpression

ALAL-1 cDNA sequence was cloned between EcoRI-BamHI sites in pcDNA3.0 vector (Invitrogen), and between EcoRI-XhoI sites in pMSCVneo retroviral vector (Clontech), for transient and stable overexpression respectively. SART3 plasmids (pCS2-SART3, pCS2-myc-SART3) were kindly provided by Michael Rape's laboratory (UC Berkeley). For transient overexpression, plasmids were transfected with Lipofectamine 2000 using manufacturers protocol.

### Nuclear-Cytoplasmic Fractionation

3×10⁶ cells were lysed in 500 μl of lysis buffer (20 mM Tris-HCl pH 7.5%, 0.1% NP-40, 280 mM NaCl, 3 mM MgCl₂ and RNasin (Promega)), and incubated on ice for 10 min. The cell lysate was then layered over 500 ul of sucrose cushion (50% sucrose in cell lysis buffer) and centrifuged at 13,000 rpm at 4° C. for 10 min. The resulting supernatant corresponded to the cytoplasmic fraction. To resuspend the nuclei pellet 500 μl of triton buffer (10 mM Tris, 100 mM NaCl, 1 mM EGTA, 300 mM sucrose, 0.5 mM NaVO3, 50 mM NaF, 1 mM phenylmethylsulphonyl fluoride, 0.5% triton X-100, protease inhibitor cocktail, and RNAsin) were added; RNA was then extracted using Trizol.

### Immunofluorescence

HCC95 cells were grown on glass coverslips and transfected with the corresponding plasmids and siRNAs. 72 hours after transfection, the cells were fixed with 4% methanol-free paraformaldehyde and were incubated with the anti-myc (Cell Signaling #2276) antibody for 30 minutes followed by incubation with the donkey anti-mouse antibody coupled to Alexa Fluor® 488 (ThermoFisher) for 30 more minutes. Coverslips were then washed three times with washing buffer (0.5% NP-40, 0.01% Na Azide, diluted in PBS), briefly air-dried, and mounted in the DAPI-containing VectaShield medium (Vector). Cells were visualized using the 40× objectives (Zeiss Axio Imager M1) and images were captured with the ZEN microscopy software.

### RNA Fish

RNA FISH was performed as previously described (Marchese et al., 2016. Mol Cell 63, 397-407), using fluoresceinlabeled Locked Nucleic Acid (LNA) DNA probes recognizing ALAL-1.

FISH probes
#1
(SEQ ID NO: 67)
ATATACCTGAGGTCTGCCAGGA
#3
(SEQ ID NO: 68)
ATCTGGGTCACCGAAACTGTA

### PBMC Isolation

Peripheral blood mononuclear cell (PBMC) were isolated from healthy volunteers by density gradient centrifugation (1.077 g ml-1) (Ficoll-Paque Plus, GE healthcare). Cells were resuspended in RPMI 1640 culture medium (GIBCO) supplemented with 10% (v/v) heat-inactivated, fetal bovine serum, penicillin-streptomycin (1%) and β-mercaptoethanol (50 μM) (Sigma).

### Cell Migration Assays

In vitro cell migration assays were performed using 24-well Trans-well chambers (5 μm pore size; Costar). 3×10⁵ of PBMC were added to the upper chamber and were incubated for 5 h with HCC95 cells transfected with siRNA Ctrl or siRNA ALAL-1 in the bottom well of the chamber. To determine the number of migratory cells, the lower cells were analyzed by flow cytometry with Perfect count microspheres (Citognos) and fluorochrome-conjugated mAbs agains: CD3-BV421 (UCHT1), CD8-PeCy7 (RPA-T8), CD4-FITC (OKT4), CD19-PE (HIB19), CD56-APC (HCD56) and CD14-BV510 (M5E2). Samples were acquired on a FACSCanto-II cytometer (BD Biosciences). Data were analyzed using FlowJo software (TreeStar).

### Cytokine Production by PBMC

2×10⁵ of PBMC were seed in 96-well plate and stimulated in vitro with human T-Activator CD3/CD28 beads (Gibco) in the presence of conditioned medium of HCC95 transfected with siRNA Ctrl or siRNA ALAL-1 and treated with TNF-α. After 48 h, IFN-γ and IL-2 secretion to the culture supernatant was measured by BD OptEIA human ELISA Set (BD Pharmingen).

### Results

Several Frequent Cancer-Associated Somatic Copy Number Alterations (SCNAs) Devoid of Protein-Coding Genes Contain lncRNAs

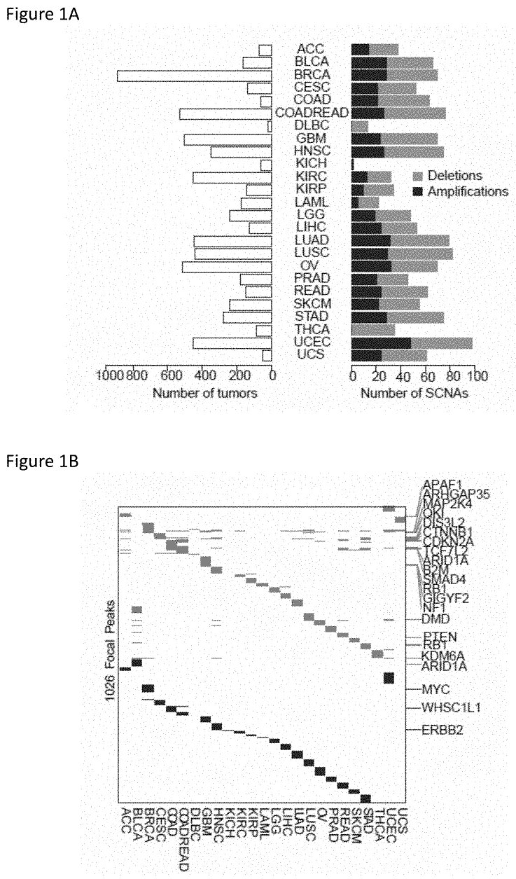

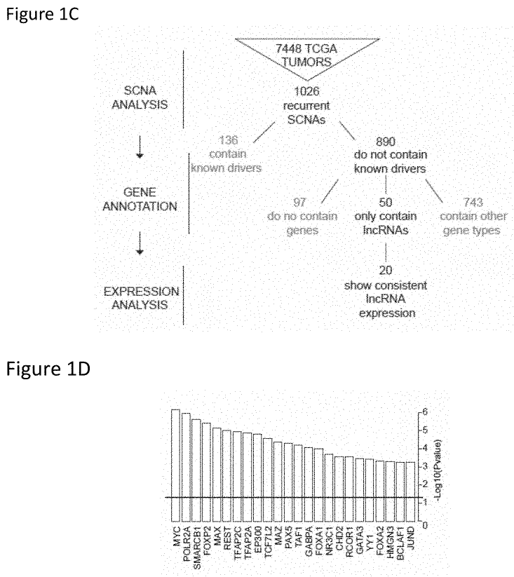

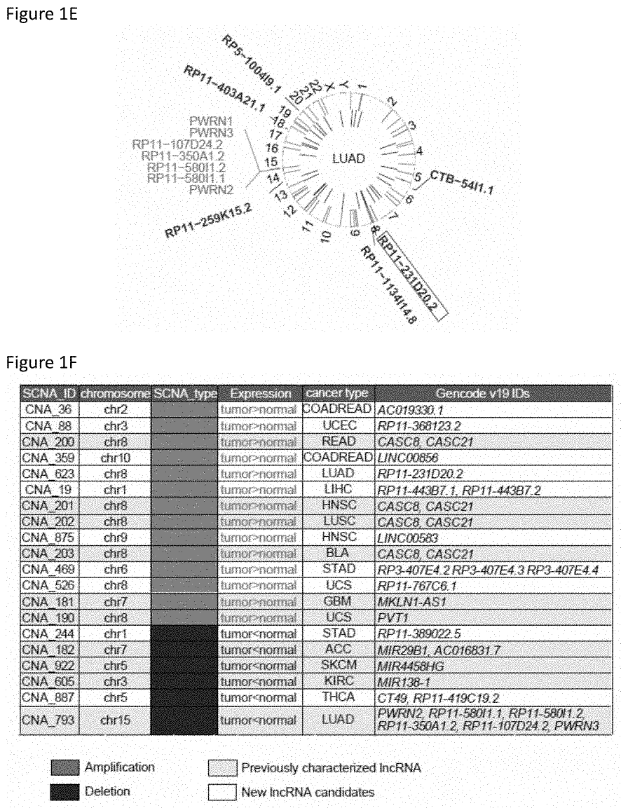

To identify those lncRNAs frequently amplified or deleted in cancer the SCNA data available from The Cancer Genome Atlas (TCGA) comprising a total of 7448 tumors of 25 different tumor types were retrieved (FIG. 1A). To detect the potentially relevant SCNAs the GISTIC 2.0 algorithm (Mermel, C. H. et al. 2011. Genome Biol. 12, R41) was used, which assigns a score to each alteration based on its amplitude (copy number changes) and frequency across all samples (G-score=Frequency×Amplitude). Q-values were then calculated using G-scores and a threshold of q-value <0.25 was established to select significant alterations. With this method 1377 SCNAs were identified, (540 amplifications and 837 deletions) at three different levels: region, enlarged peaks and focal peaks. While genomic instability inherent to cancer cells can lead to large SCNAs that contain thousands of genes (regions and enlarged peaks), driver alterations only usually target few genes, accordingly, the rest of the analysis on SCNAs was focused at the focal peak level. In total, 1026 unique copy number-altered focal regions were identified. 916 of them were specific to a tumor type, while the rest (110) were present in several tumor types (FIG. 1B).

To have a comprehensive view of all genes, both coding and non-coding affected by the copy number alterations, the SCNAs were classified based on the annotation of genes contained in them (GENCODE v19), as well as taking into consideration their known cancer driver features (FIGS. 1B-C). Out of the 1026 SCNAs, 136 contained a known cancer driver as defined by the high-confidence driver list generated by combining five different methods for the identification of protein-coding genes exhibiting several signals of positive selection (Tamborero, D. et al. 2013. Sci Rep 3, 2650) (FIG. 1C). For instance, the tumor suppressors PTEN and RB1 were found in frequently lost regions, while the oncogene MYC was inside a frequently amplified SCNA, confirming the validity of the SCNA analysis to pinpoint genes relevant to tumor progression. On the other hand, the 890 remaining SCNAs did not contain any known cancer driver gene. The classification of these SCNAs based on the biotypes of their genes, showed that 50 only contain lncRNAs (FIG. 1C), thus these lncRNAs are frequently lost or amplified independently of protein-coding genes, suggesting that they could act as cancer drivers. In addition, 97 SCNAs did not contain any annotated gene, and were therefore classified as ‘gene deserts’.

lncRNAs Inside Frequent SCNAs have Functional Features

Next, to increase the insight into the functional characteristics of the lncRNAs within frequent SCNAs, their regulation was analyzed by relevant transcription factors. To that end, the transcription start sites (TSS) of the copy number altered lncRNAs were retrieved and their regulatory region 1 Kb upstream and 1 Kb downstream of the TSS was arbitrarily defined. The resulting genomic coordinates were then intersected with the binding sites of 161 transcription factors (TFs) obtained from Chromatin Immunoprecipitation (ChIP-seq) experiments reported by ENCODE. The regulatory regions of deleted lncRNAs showed some enrichment in binding sites for POUF51/OCT4 (p-value=0.0080) and NANOG (p-value=0.0384), indicating regulation by pluripotency-related transcription factors. Interestingly, amplified lncRNAs were enriched for oncogenic factors such as MYC (p-value=6.93e⁻⁷), MAX (p-value=6.97e⁻⁶) and JUND (p-value=0.0005201) (FIG. 1D), pointing towards their specific regulation by oncogenic signals in line with their amplified status.

If the genes contained within SCNAs have an impact in cancer progression, they should present a change in expression in tumors consistent with the sense of the copy number alteration (amplification or deletion). The SCNA results were cross-compared with RNA expression profiling analysis in cancer from publicly available RNA-seq data. The comprehensive annotation of cancer-associated lncRNAs (miTRANSCRIPTOME, Iyer, M. K. et al. 2015. Nat Genet 47, 199-208) was used, which identified additional lncRNAs in 13 amplified regions where no gene was previously annotated by GENCODE v19. The final analysis pinpointed a total of 20 putative functional alterations (14 amplifications and 6 deletions) in which significant expression changes of the contained long non-coding RNAs are present in tumor samples compared to normal tissue (FIG. 1F). Among the regions selected by this method we found some previously characterized cancer-related lncRNAs such as PVT1, localized downstream from the MYC locus, or CARLo-5 located in an amplified region upstream of MYC locus. However, most of the identified lncRNAs remain uncharacterized, and so far no functional role has been assigned to them.

### ALAL-1 is a Potential Driver of Non-Small Cell Lung Cancer

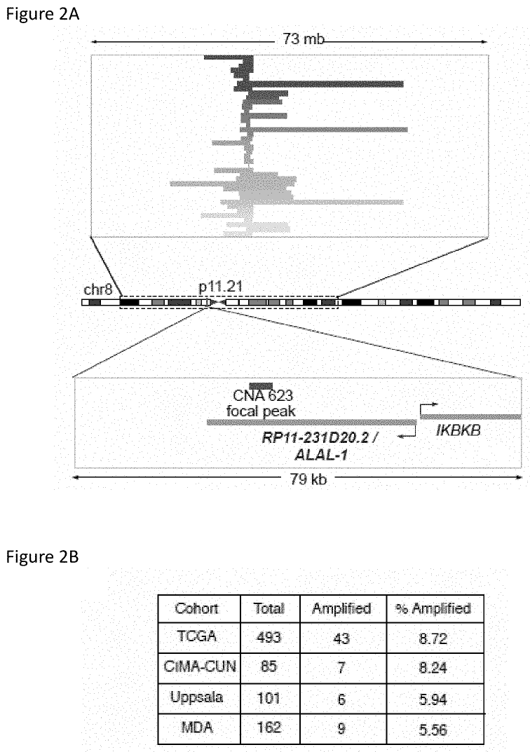

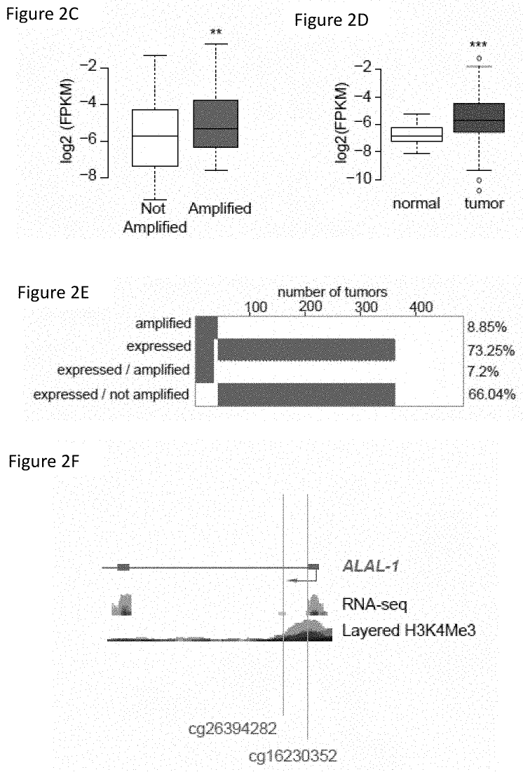

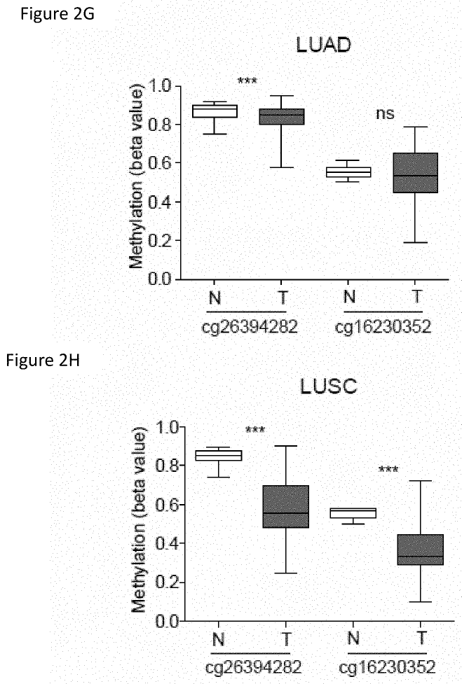

To further study the role of the lncRNAs identified by the SCNA analysis, it was decided to focus on those found in lung cancer, the leading cause of cancer-related death worldwide. The inspection of these SCNAs (CNA_202, CNA_623 and CNA_793, FIGS. 1E-F) showed that CNA_202 contains CARLo-1/CASC8, previously related to several types of tumors. The loci affected by CNA_793 have also been linked to lncRNAs, since it maps to the frequently deleted Prader Willis/Angelman region containing 7 non-coding RNAs (PWRN2, RP11-580I1.1 RP11-580I1.2, RP11-350A1.2, RP11-107D24.2, PWRN3 and PWRN1). On the other hand, CNA_623 only overlaps the uncharacterized RP11-231D202.2 (FIG. 2A).

First, it was determined whether the amplification of CNA_623 detected in the TCGA cohort was also found in independent cohorts of lung cancer patients. Indeed, while the analysis of the TCGA lung adenocarcinoma (LUAD) cohort showed that 43/493 (8.7%) samples contain this alteration, the amplification was consistently validated in three additional independent lung adenocarcinoma tumor cohorts: CIMA-CUN, MD Anderson (Aramburu, A. et al. BMC Genomics. (2015). 16, 752) and Uppsala (Micke, P. et al. J Thorac Oncol (2011).6, 1833-40) with amplification in 8.24, 5.56 and 5.94% of the tumors respectively (FIG. 2B).

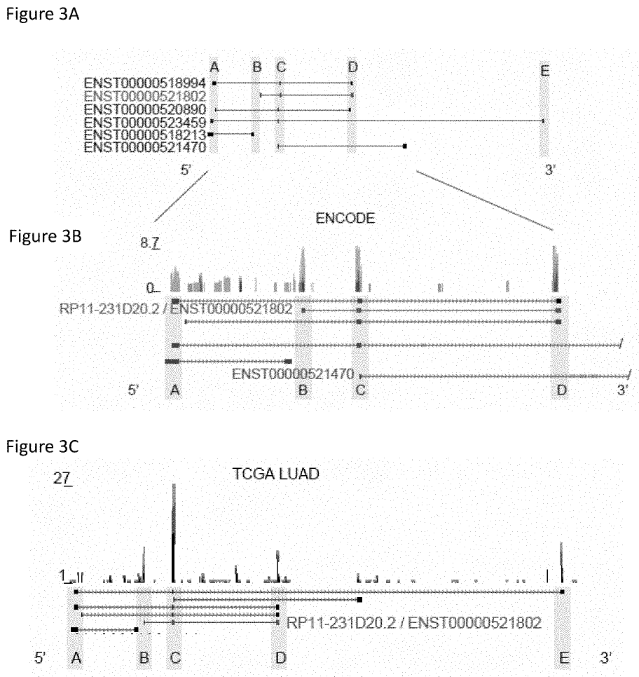

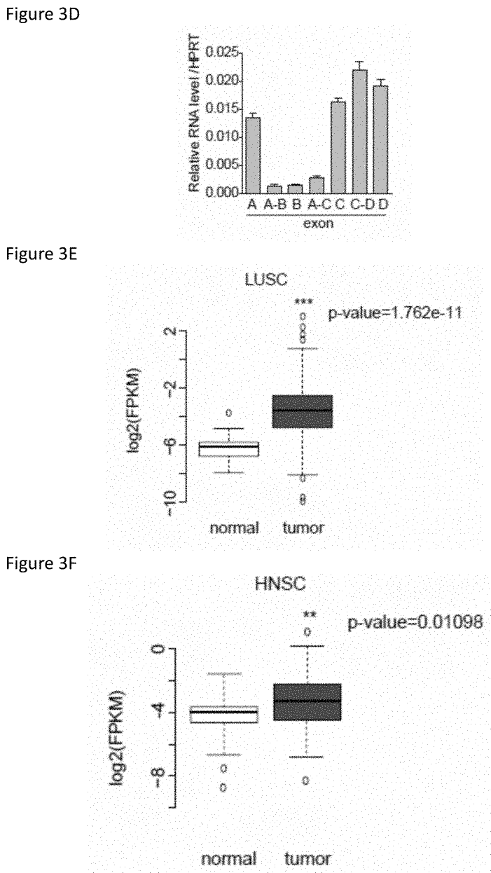

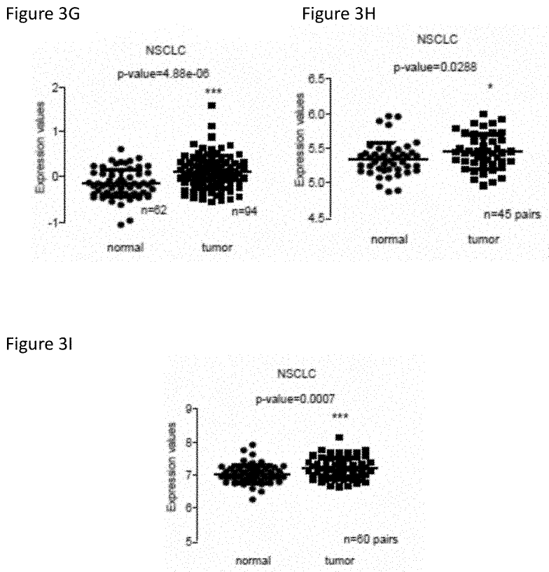

CNA_623 uniquely maps to RP11-231D20.2, hereon referred to as Amplified lncRNA Associated with Lung Cancer-1 (ALAL-1). ALAL-1 is located between Plasminogen Activator Tissue Type (PLAT) and Inhibitor of Nuclear Factor kappa B Kinase Subunit Beta (IKKβ/IKBKB) genes, being a divergent antisense transcript of IKKβ. GENCODE v19 annotation shows six different transcripts associated to ALAL-1 locus (FIGS. 3A-B). In order to assess which of the transcriptional forms is predominantly expressed in lung tumor cells, a TCGA-LUAD sample with a mean expression of ALAL-1 was selected, and the RNA-seq reads mapped to the locus (FIG. 3C). The RNA-seq supported the predominant expression of the 415 nt-long transcript form ENST00000521802 (FIG. 3C), which has three exons, observation confirmed by qRT-PCR using different sets of primers (FIG. 3D). The subsequent study was focused on this transcript form of ALAL-1.

To evaluate the possible role of ALAL-1 in lung cancer, it was tested whether ALAL-1 was indeed overexpressed in the samples where the amplification was present. To do this, the samples were classified in two groups: with and without amplification (n=43 and n=322 respectively). It was observed significantly higher expression of ALAL-1 in the amplified group (FIG. 2C), moreover the expression analysis of all the TCGA-LUAD samples (comparing normal vs tumor) also showed a significant difference on ALAL-1 expression levels (FIG. 2D). In fact, around 66% of the tumor samples with elevated expression of ALAL-1 lacked the amplification of ALAL-1 locus (FIG. 2E). Moreover, ALAL-1 was also overexpressed in additional cohorts and tumor types: lung squamous carcinoma (LUSC) and head and neck squamous carcinoma (HNSC) (FIGS. 3E-I). The most significant difference in ALAL-1 expression comparing tumor versus normal samples was observed in the lung squamous carcinoma (LUSC) cohort (p-value=1.762e⁻¹¹) where no frequent amplification of ALAL-1 was identified, suggesting that other mechanisms besides amplification could be involved in ALAL-1 overexpression. To investigate this possibility, the DNA methylation of ALAL-1 locus was analyzed in lung cancer patient samples, which led to the detection of two differentially hypomethylated CpGs mapping to the 5′ end of ALAL-1 in the LUAD cohort (cg cg26394282 p-value=1.76e⁻¹¹) (FIGS. 2F-G), but even more significant in the LUSC tumor cohort (cg26394282 p-value=1.89e⁻²⁴, cg16230352 p-value=3.05e⁻¹⁹) (FIG. 2H), suggesting that hypomethylation could explain the observed overexpression of ALAL-1 in this tumor type.

Together these data indicate that ALAL-1 is as an overexpressed lncRNA, targeted by genetic and epigenetic mechanisms, that has a role as an oncogene in non-small cell lung cancer pathogenesis.

### ALAL-1 Promotes the Oncogenic Phenotype of Lung Cancer Cells

In order to experimentally test the potential oncogenic role of ALAL-1 it was set to identify lung cancer cell lines with a genetic background similar to the one present in the tumor samples (i.e. amplification of ALAL-1). For this, The Cancer Cell Line Encyclopedia (CCLE) was interrogated, where around 12% of the lung cancer cell lines bear amplification of ALAL-1 (FIG. 4A). Among them, HCC95 (LUSC) and H1648 (LUAD) cell lines showed the highest level of amplification of the ALAL-1 locus (FIG. 4B), amplification that we independently estimated as ˜8 copies per cell (FIG. 4C). Moreover, the expression level of ALAL-1 RNA was also increased, correlative to the amplification of the gene, and significantly higher compared to lung cancer cell lines without ALAL-1 amplification (A549 and H2170) or to non-tumoral cells (FIG. 4D).

To test the effect of ALAL-1 genomic amplification it was set to revert it. To do so, the genome editing technology CRISPR/Cas9 was applied to HCC95 cells using two single guide RNAs (sgRNAs) flanking exon D of ALAL-1, obtaining a deletion of ˜500 bp (FIG. 4E). All the clones recovered with the intended deletion were heterozygous (FIG. 4F), due to the presence of multiple copies of the gene and the low frequency of several editing events occurring in the same cell. Nevertheless, the obtained heterozygous clones had a concomitant reduction of ALAL-1 RNA levels (FIG. 4G). Interestingly, these clones presented reduced in vitro proliferation (FIG. 4H), reduced colony formation capacity and increased apoptosis, (FIG. 4I and FIGS. 5A-B), as well as reduced tumor growth capacity in xenograft mouse models (FIG. 4J).

While the genomic deletion of ALAL-1 indicates a role of the gene in cancer cells, it doesn't allow determining whether the observed effect is due to the removal of the DNA sequence or to the decrease of ALAL-1 RNA levels. In order to uncouple the function of the lncRNA from that of the underlying genomic elements, siRNA knockdown experiments were carried out with two different siRNAs targeting ALAL-1, which reduced the levels of ALAL-1 leaving the ALAL-1 amplified locus intact (FIG. 4K). Similarly to the observed after genomic deletion, the inhibition of ALAL-1 not only affected the in vitro proliferative and colony formation capacity and apoptosis levels of HCC95 cells (FIGS. 6A-C), but also impaired their in vivo tumor formation capacity (FIGS. 4L-M). Comparable effects were observed when ALAL-1 was inhibited in H1648 cells (FIGS. 6D-F). Conversely, the overexpression of ALAL-1 in HCC95, H1648 and A549 lung cancer cell lines resulted in an increased clonogenic capacity (FIGS. 6G-O).

Taken together, data show that ALAL-1 is a functional lncRNA with a pro-oncogenic role in lung cancer.

### ALAL-1 is a Regulator of the NF-κB Pathway

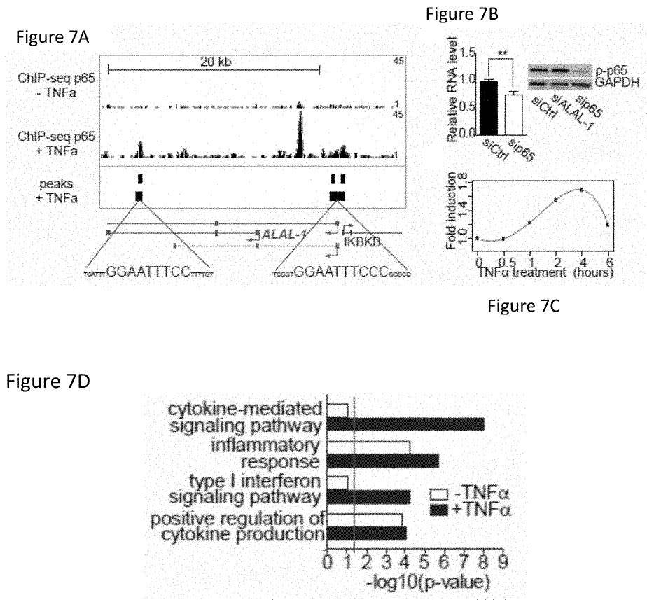

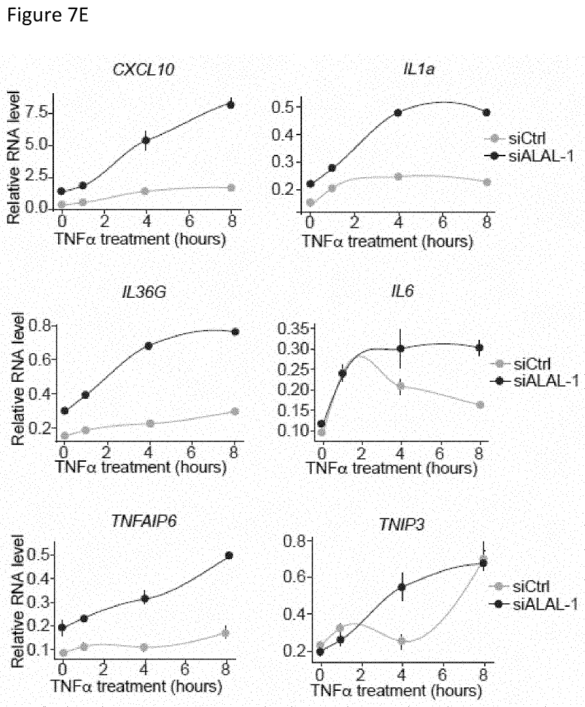

To gain further understanding of ALAL-1 cellular function, its transcriptional regulation was investigated. The analysis of ALAL-1 genomic locus identified p65/RelA consensus binding sites around its transcriptional start site (TSS) (FIG. 7A). Consistently with this, it was observed the presence of ChIP-seq peaks corresponding to p65/RelA subunit of NF-κB in different cell types (HUVEC, A549 and IMR90 (FIG. 7A). The association of p65/RelA was detected upon TNFα treatment (FIG. 7A), similar to other NF-κB bona-fide target genes. Also in agreement with this, the levels of ALAL-1 were reduced in conditions of p65/RelA knockdown (FIG. 7B), while it was induced by TNFα, with a peak of induction after 4 hours of treatment (FIG. 7C). These results demonstrate that ALAL-1 is a direct target of NF-κB and it is induced upon TNFα treatment.

In order to better understand the role of ALAL-1 in cancer cells, the changes in gene expression caused by the lncRNA inhibition were explored. Since ALAL-1 is induced by TNFα, RNA-seq analysis on HCC95 cells subjected to ALAL-1 knockdown or transfected with a control siRNA and untreated or treated with TNFα was performed.

The downregulation of ALAL-1 in untreated cells affected the expression of 116 genes, while in TNFα-treated cells 108 genes were altered (Adj. p-value <0.01). The most significantly affected cellular pathways were those related with the inflammatory and immune responses, and the induction of cytokines in the context of the NF-κB pathway, even to a higher extent when the cells were treated with TNFα (FIG. 7D). Consistently with this observation, the induction of the inflammatory response by TNFα treatment of HCC95 cells caused reduced survival and increased apoptosis (FIG. 8A), phenotype similar to that caused by ALAL-1 inhibition in the tumor cell line (FIGS. 5A and 6C). Moreover, the genes with expression most highly correlated with that of ALAL-1 in the CCLE dataset indicated enrichment in Gene Ontologies such as regulation of cell proliferation, NF-κB cascade and cytokine activity, suggesting a role of ALAL-1 in these processes (FIG. 8B). Among the genes affected by ALAL-1 inhibition are several chemokines and cytokines such as CXCL-10, CXCL-11, IL36G and IL1-α, as well as other regulators of the immune response like MEFV, and TNFAIP6, which were independently validated (FIG. 7E). Similar expression changes were observed in the cell lines where ALAL-1 was deleted by CRISPR-Cas9-mediated gene edition (FIG. 8C, confirming that they are dependent on ALAL-1. However ALAL-1 neighbor genes PLAT and IKBKB were not affected, indicating that ALAL-1 doesn't regulate their expression.

Together, data show that ALAL-1 is activated by NF-κB, acting in turn as a negative modulator of the inflammatory response, resulting in an autonomous effect in the proliferation of the tumor cells.

### ALAL-1 Regulates the NF-κB Pathway Through SART3 and USP4

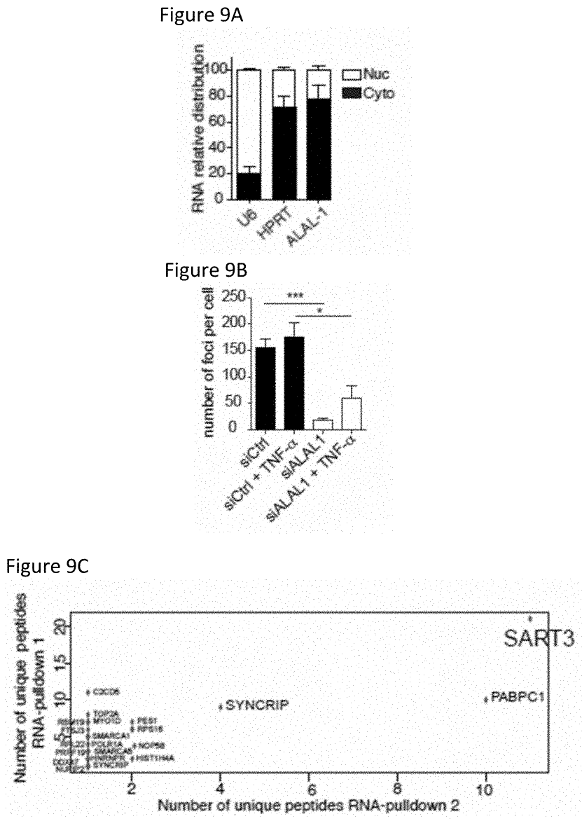

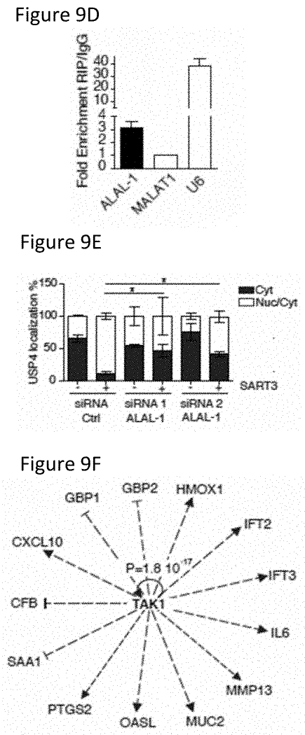

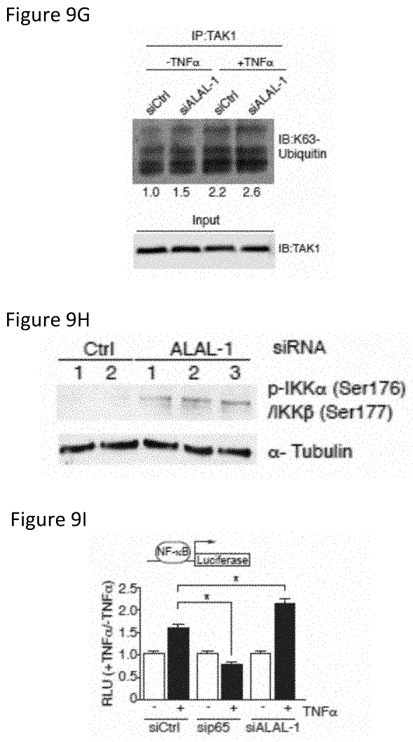

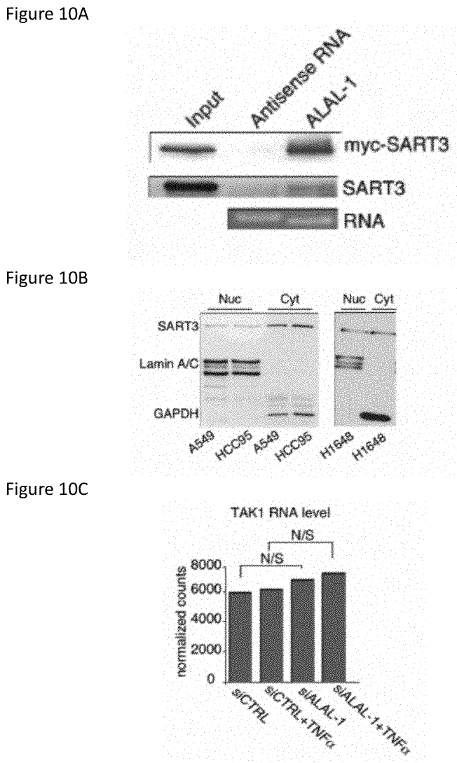

Subcellular fractionation and RNA FISH experiments showed that the localization of ALAL-1 in the lung cancer cells is predominantly cytoplasmic, with more than 80% of the lncRNA present in the cell cytoplasm (FIG. 9A). In addition, the quantification by independent methods estimated that HCC95 cells express more than 150 molecules of ALAL-1 per cell (FIG. 9B). We therefore hypothesized that ALAL-1 could be acting in the cytoplasm through the interaction with proteins. In order to identify the interacting partners of ALAL-1 we performed in vitro RNA pulldown experiments using as control the antisense RNA, with the same length but unrelated sequence. The differential protein bands were then cut and subjected to mass spectrometry. These analyses showed that the protein that specifically interacts with ALAL-1 with the highest number of peptides is Squamous cell carcinoma antigen recognized by T cells (SART3), also referred as HIV-Tat interacting protein (Tip110) (FIG. 9C). This interaction was confirmed in several independent experimental replicates (FIG. 10A), as well as by co-immunoprecipitation of the endogenous ALAL-1 with SART3 protein (FIG. 9D).

Several functions have been reported for SART3. While it is known to act in the nucleus as a U6 recycling factor during splicing, it is also found in the cytoplasm of proliferating cells. Indeed, we observed the cytoplasmic localization of SART3 in the lung cancer cell lines (FIG. 10B), consistent with the interaction between SART3 and ALAL-1 in this cellular compartment. In the cytoplasm SART3 interacts with the ubiquitin specific proteases USP4 and USP15. USP4 is a key regulator of the NF-κB pathway through the deubiquitination of several proteins that are polyubiquitinated upon TNFα treatment. It was therefore hypothesized that the interaction between ALAL-1 and SART3 could affect USP4 function, and in consequence the NF-κB signaling. It was first analyzed USP4 shuttling between the nucleus and the cytoplasm, which is known to be regulated by SART3. As previously described, the overexpression of SART3 resulted in nuclear translocation of USP4, but remarkably this only occurred when ALAL-1 was present. In contrast, ALAL-1 inhibition prevented the translocation of USP4 mediated by SART3 (FIG. 9E), indicating that ALAL-1 affects USP4 relocalization. Similarly, it was reasoned that the activity of USP4 on its targets could be dependent on ALAL-1. Interestingly, the in silico analysis of the gene changes caused by ALAL-1 inhibition predicted that their most significant upstream regulator is the kinase TAK1, major target of USP4 in the NF-κB pathway (p-value=3.2 10⁻¹⁰ in untreated and p-value=1.8 10⁻¹⁷ in TNFα-treated cells) (FIG. 9F), while TAK1 mRNA levels were not affected by ALAL-1 (FIG. 10C). It was then analyzed the ubiquitination levels of TAK1 in conditions of ALAL-1 knockdown compared to control with or without TNFα treatment. As expected, TNFα caused an increase in the levels of TAK1 ubiquitination, but these levels were further increased when ALAL-1 was inhibited (FIG. 9G), in agreement with a reduced activity of USP4 over TAK1. Moreover, and also consistently with this observation, the phosphorylation of IKKB, which is catalyzed by TAK1, was increased upon ALAL-1 inhibition (FIG. 9H), which in turn resulted in the activation of NF-κB transcriptional activity, the downstream event of the signaling cascade (FIG. 9I).

These results indicate that ALAL-1 regulates the activation of NF-κB pathway through its interaction with SART3, modulating the ubiquitination of TAK1 by USP4.

### ALAL-1 Contributes to the Immune Evasion of Non-Small Cell Lung Squamous Tumors

The gene signature regulated by ALAL-1 includes an important number of immune cytokines, which are significantly induced upon ALAL-1 inhibition (FIG. 7E). Among them are CXCL-10 and CXCL-11, chemokines involved in Th1 response and Th1, CD8, and NK trafficking, the strong mediator of inflammation IL1-α or IL36G, which is known to change the tumor microenvironment positively for tumor rejection. Taking into consideration the critical role that the interaction between the tumor and its microenvironment plays in cancer progression, it was decided to investigate the relationship between ALAL-1 and the immune environment of the tumor. To determine whether there is a correlation between ALAL-1 and the level of infiltration of tumors by immune populations, TCGA lung squamous carcinoma tumor samples were grouped in six subgroups ranging from lower to higher presence of cytotoxic cells, based on the enrichment of gene signatures of specific immune cell populations (FIG. 11A). The expression of ALAL-1 was significantly elevated in tumors with lower levels of immune infiltration, while the tumors with the highest levels of cytotoxic infiltration expressed the lowest levels of ALAL-1 (FIG. 11A). In order to confirm this observation the expression of ALAL-1 was quantified in an independent cohort of lung squamous carcinoma patients and determined the presence of PD-1 positive infiltrating cells, which is related to the strength of T cell receptor signaling, and thus to the functional avidity of specific T cells. In agreement with our previous data, the tumors with lower levels of PD-1 positive cells expressed higher levels of ALAL-1 and vice versa (FIG. 11B). These results indicate that in vivo ALAL-1 contributes to inhibit the infiltration of the tumor by immune populations.

If ALAL-1 expression contributes to the immune evasion of the tumor, the inhibition of the lncRNA could in principle facilitate the attraction and activation of cytotoxic immune populations. To test this hypothesis, HCC95 cells transfected with ALAL-1 siRNA or control siRNA were incubated, and assayed their influence on the migration of cells from peripheral blood (FIG. 11C). The inhibition of ALAL-1 resulted in the increased migration of CD8+ T lymphocytes, CD19+ B lymphocytes, NK cells and monocytes towards the tumor cells (FIG. 11C). Moreover, pre-activated lymphocytes incubated with media conditioned by the ALAL-1-depleted cells, showed higher percentage of PD-1 positive cells, as well as elevated production of IFNγ and IL-2 cytokines (FIGS. 11D-E). Together, these data indicate that the inhibition of ALAL-1 can enhance the activity of cytotoxic immune cells with a potential therapeutic anti-tumor effect. Thus an oncogenic lncRNA that mediates cancer immune evasion has been unveiled, pointing to a new target for immune potentiation.

1. An inhibitor of functional expression of AC083973.1 gene, wherein the inhibitor is selected from (i) a nucleic acid that specifically binds to the AC083973.1 gene or to the transcriptional product of said gene blocking the expression of said gene and (ii) a nuclease that specifically binds and enzymatically inactivates said gene.

2. The inhibitor according to claim 1 wherein the inhibitor is selected from a sgRNA, a DNA polynucleotide that can be transcribed into a sgRNA, siRNA, shRNA, miRNA, an antisense oligonucleotide, and/or a ribozyme.

4. The inhibitor according to claim 2, wherein the sgRNA is selected from SEQ ID NO: 69 and SEQ ID NO: 70, the DNA polynucleotide that can be transcribed into a sgRNA is selected from SEQ ID NO: 1 and SEQ ID NO: 3, and/or the siRNA is selected from SEQ ID NO: 10 and SEQ ID NO: 11.

5. A pharmaceutical composition comprising an effective amount of the inhibitor according to claim 1.

6. The pharmaceutical composition according to claim 5 further comprising a compound selected from a CTLA-4 inhibitor, a PD-1 inhibitor and a PD-L1 inhibitor.

7. A method of treating a tumor in a subject comprising administering to the subject a therapeutic effective amount of the inhibitor according to claim 1, wherein the tumor is characterized by having increased copy number of the AC083973.1 gene, and/or decreased methylation of said gene and/or increased levels of the transcriptional product of said gene compared to a reference value.

8. The method according to claim 7, wherein decreased methylation is detected in a CpG selected from the group consisting of cg26394282 and cg16230352.

9. The method according to claim 7, wherein the transcriptional product of said gene is the transcript of sequence SEQ ID NO: 16.

10. The method according to claim 7, wherein the inhibitor is selected from a sgRNA, a DNA polynucleotide that can be transcribed into a sgRNA, siRNA, shRNA, miRNA, an antisense oligonucleotide, and/or a ribozyme.

11. The method according to claim 10, wherein the sgRNA is selected from SEQ ID NO: 69 and SEQ ID NO: 70, the DNA polynucleotide that can be transcribed into a sgRNA is selected from SEQ ID NO: 1 and SEQ ID NO: 3, and/or the siRNA is selected from SEQ ID NO: 10-11.

12. The method according to claim 7, wherein the tumor is a lung cancer or head and neck cancer.

13. The method according to claim 12, wherein the lung cancer is non-small cell lung cancer.

14. The method according to claim 13, wherein the non-small cell lung cancer is squamous cell carcinoma or adenocarcinoma.

15. The method according to claim 14, wherein the head and neck cancer is head and neck squamous cell carcinoma.

16. A method of diagnosing and treating a tumor in a subject comprising:
(i) determining the copy number of the AC083973.1 gene, and/or the methylation level of said gene and/or the level of the transcriptional product of said gene in a sample,
(ii) comparing the copy number and/or methylation level and/or level obtained under (i) with a reference value,
(iii) diagnosing the subject as having a tumor susceptible of being treated by the inhibitor of functional expression of AC083973.1 according to claim 1 when the copy number of the gene is increased and/or the methylation level is decreased and/or the level of the transcriptional product of the gene is increased compared to the reference value and
(iv) administering the subject diagnosed with the tumor with a therapeutic effective amount of an inhibitor of functional expression of AC083973.1 according to claim 1.

17. The method according to claim 16, wherein decreased methylation is detected in a CpG selected from the group consisting of cg26394282 and cg16230352.

18. The method according to claim 16, wherein the transcriptional product of said gene is the transcript of sequence of SEQ ID NO: 16.

19. The method according to claim 16, wherein the inhibitor is selected from a sgRNA, the DNA polynucleotide that can be transcribed into a sgRNA, siRNA, shRNA, miRNA, an antisense oligonucleotide, and/or a ribozyme.

20. The method according to claim 18, wherein the sgRNA is selected from SEQ ID NO: 69 and SEQ ID NO: 70, the DNA polynucleotide that can be transcribed into a sgRNA is selected from SEQ ID NO: 1 and SEQ ID NO: 3, and/or the siRNA is selected from SEQ ID NO: 10-11.

21. The method according to claim 16, wherein the sample is a tumor sample.

22. The method according to claim 16, wherein the cancer is lung cancer or head and neck cancer.

23. The method according to claim 22, wherein the lung cancer is non-small cell lung cancer.

24. The method according to claim 22, wherein the non-small cell lung cancer is non-small squamous cell carcinoma or adenocarcinoma.

25. The method according to claim 22, wherein the head and neck cancer is head and neck squamous cell carcinoma.

26. A method for selecting a therapy for treating a subject with a tumor comprising:
(i) determining the copy number of the AC083973.1 gene, and/or the methylation level of said gene and/or the level of the transcriptional product of said gene in a sample,
(ii) comparing the copy number and/or methylation level and/or level obtained under (i) with a reference value,
(iii) selecting a therapy consisting of an inhibitor of functional expression of AC083973.1 according to claim 1 if the copy number of the gene is increased and/or the methylation level is decreased and/or the level of the transcriptional product of the gene is increased compared to the reference value.

27. A method for increasing the migration of cytotoxic immune cells towards a tumor comprising administering the inhibitor according to claim 1 to a subject suffering from said tumor, wherein the tumor has increased copy number of the AC083973.1 gene and/or decreased methylation of said gene and/or increased level of the transcriptional product of said gene compared to a reference value.


SEQUENCE LISTING (70 sequences)

SEQ ID NO: 1 - DNA, 25 bp, Artificial sequence
  Synthetic Construct
caccgttcccagatggggcgacggc

SEQ ID NO: 2 - DNA, 25 bp, Artificial sequence
  Synthetic Construct
aaacgccgtcgccccatctgggaac

SEQ ID NO: 3 - DNA, 25 bp, Artificial sequence
  Synthetic Construct
caccgtatattgctaagacgttgac

SEQ ID NO: 4 - DNA, 25 bp, Artificial sequence
  Synthetic Construct
aaacgtcaacgtcttagcaatatac

SEQ ID NO: 5 - DNA, 22 bp, Artificial sequence
  Synthetic Construct
ttgatgtccccaatatacctga

SEQ ID NO: 6 - DNA, 22 bp, Artificial sequence
  Synthetic Construct
tgtggtttctttatgtggttgc

SEQ ID NO: 7 - DNA, 18 bp, Artificial sequence
  Synthetic Construct
tgcccactgtctggcaaa

SEQ ID NO: 8 - DNA, 19 bp, Artificial sequence
  Synthetic Construct
ccaccatgcttggcactat

SEQ ID NO: 9 - RNA, 20 nt, Artificial sequence
  Synthetic Construct
cagucgcguuugcgacuggc

SEQ ID NO: 10 - RNA, 19 nt, Artificial sequence
  Synthetic Construct
gguauauuggggacaucaa

SEQ ID NO: 11 - RNA, 19 nt, Artificial sequence
  Synthetic Construct
ggauauggagaaaauuauu

SEQ ID NO: 12 - RNA, 19 nt, Artificial sequence
  Synthetic Construct
gcccuaucccuuuacguca

SEQ ID NO: 13 - DNA, 815 bp, Homo sapiens
gcgactcccccggggcgggggtggcgtgtggtctctagacaactgagggaaagtagacagaccgtgctcaccgatttaaaagaacagccctgaaatgacccccgttacaccctgttctctttagacaagttacttccacctggagcggtttctgggcccgggtatggggatttcttcacccggccccctaggctttcacctcggtggaatttcccgcgcccccgctgggatgatttagggactgttggctagaactgcccttgcctaccgccctactgcgggcgctcccctgtctggggggtgcttggctgggtgtggagaaggatcaatcacagctgactcctcagaggtgacagcccctgcccccttaaaaacaagaaaagacctagcagtgtgaagccaaactgcacccccagccccggtgacatgaggaaaccgtgaggcgctcagcctgaggttcatcgttcatctccttcctgcttggaatcagaatgtcctcactgctgagtttctccgttttatgggattacattagtggacaaaggagtgaagcaaagcagcactgagctcctccaagtaaagaaagaattgttttctgtacaacctggagctgaatcatagaaggagaagacagcttgaaagataagaggccgtaaggcagaccacagaggatggggaggatttccccagcaccccagcccccatagtcagctctggttcagcgatttcagcaactctttcatgacccatctgattttgttttatcccagccctgcagagaaaggactctgcaggtcaggacttgcttcttcaac

SEQ ID NO: 14 - DNA, 601 bp, Homo sapiens
gcgggcgctcccctgtctggggggtgcttggctgggtgtggagaaggatcaatcacagctgactcctcagaggtgacagcccctgcccccttaaaaacaagaaaagacctagcagtgtgaagccaaactgcacccccagccccggtgacatgaggaaaccgtgaggcgctcagcctgaggttcatcgttcatctccttcctgcttggaatcagaatgtcctcactgctgagtttctccgttttatgggattacattagtggacaaaggaggaagtcccagccggaaacacagaaaggatatggagaaaattattattctgcccctacagtttcggtgacccagatgggatctctgggacaccctgcctgctctacaattgggatccctggaaaagtgacagaatctggcaacagatgagtaagaaaggtcacttcctggcagacctcaggtatattggggacatcaagaaaagaataattcatcaaagtctacagatattgcaggcaaagttgaaacaagccagacaagttctctttgtattcttgagagacttggaaagtctaacctgagattccttattaaaagttccagcaaaacaga

SEQ ID NO: 15 - DNA, 590 bp, Homo sapiens
cccctgtctggggggtgcttggctgggtgtggagaaggatcaatcacagctgactcctcagaggtgacagcccctgcccccttaaaaacaagaaaagacctagcagtgtgaagccaaactgcacccccagccccggtgacatgaggaaaccgtgaggcgctcagcctgaggttcatcgttcatctccttcctgcttggaatcagaatgtcctcactgctgagtttctccgttttatgggattacattagtggacaaaggaggaagtcccagccggaaacacagaaaggatatggagaaaattattattctgcccctacagtttcggtgacccagatgggatctctgggacaccctgcctgctctacaattgggatccctggaaaagtgacagaatctggcaacagcccccacctaggaactgactcagagcaagaagacagctccgactccctgtgatttcatccctgaccaatcagctcttctagctcactggcttcccccaacccacgaagttgtccttaaaaactctgctcccgactgctgggagagactgatttgagtaataataaaactggtctcctgcacagcc

SEQ ID NO: 16 - DNA, 415 bp, Homo sapiens
gtcatgaaagggcagctgccacctcaggccatggtggcggagacaaaggctatgggccggtgtagacgtctaagaaaccaacaggaagtcccagccggaaacacagaaaggatatggagaaaattattattctgcccctacagtttcggtgacccagatgggatctctgggacaccctgcctgctctacaattgggatccctggaaaagtgacagaatctggcaacagatgagtaagaaaggtcacttcctggcagacctcaggtatattggggacatcaagaaaagaataattcatcaaagtctacagatattgcaggcaaagttgaaacaagccagacaagttctctttgtattcttgagagacttggaaagtctaacctgagattccttattaaaagttccagcaaaacaga

SEQ ID NO: 17 - DNA, 388 bp, Homo sapiens
gacccagatgggatctctgggacaccctgcctgctctacaattgggatccctggaaaagtgacagaatctggcaacaggggcctgctctgtcgcccaggctggagtgcagtggcacaaacacagcggactgcagccttgacctcccaggctcatgcaaccctcccacctcggcctctcaagaaactgggaccacaggcacgtgccatcatacctggccaatttttcagtttttttgtagagatggggtcccctgtattgctcaggctggtctcaaactcctgggctcaagtgatcctcctgtcttggtttcccaaagtgctgggattgcaggtgtgagctactgcacccggcccaataaaaaaatctcgttctgtgaaagaaatgttt

SEQ ID NO: 18 - DNA, 368 bp, Homo sapiens
tccaactcctgggctcaagtgattctcctgactacaggaagtcccagccggaaacacagaaaggatatggagaaaattattattctgcccctacagtttcggtgacccagatgggatctctgggacaccctgcctgctctacaattgggatccctggaaaagtgacagaatctggcaacagatgagtaagaaaggtcacttcctggcagacctcaggtatattggggacatcaagaaaagaataattcatcaaagtctacagatattgcaggcaaagttgaaacaagccagacaagttctctttgtattcttgagagacttggaaagtctaacctgagattccttattaaaagttccagcaaaacaga

SEQ ID NO: 19 - DNA, 22 bp, Artificial sequence
  Synthetic Construct
ttgatgtccccaatatacctga

SEQ ID NO: 20 - DNA, 22 bp, Artificial sequence
  Synthetic Construct
tgtggtttctttatgtggttgc

SEQ ID NO: 21 - DNA, 19 bp, Artificial sequence
  Synthetic Construct
agccagccagggcttactc

SEQ ID NO: 22 - DNA, 20 bp, Artificial sequence
  Synthetic Construct
ctgaggtcaacctgctcaca

SEQ ID NO: 23 - DNA, 22 bp, Artificial sequence
  Synthetic Construct
gacagaatctggcaacagatga

SEQ ID NO: 24 - DNA, 22 bp, Artificial sequence
  Synthetic Construct
ttgatgtccccaatatacctga

SEQ ID NO: 25 - DNA, 19 bp, Artificial sequence
  Synthetic Construct
agccggaaacacagaaagg

SEQ ID NO: 26 - DNA, 20 bp, Artificial sequence
  Synthetic Construct
gggatcccaattgtagagca

SEQ ID NO: 27 - DNA, 20 bp, Artificial sequence
  Synthetic Construct
aaaggtcacttcctggcaga

SEQ ID NO: 28 - DNA, 23 bp, Artificial sequence
  Synthetic Construct
ggaatctcaggttagactttcca

SEQ ID NO: 29 - DNA, 18 bp, Artificial sequence
  Synthetic Construct
aatggagcaggctgtgga

SEQ ID NO: 30 - DNA, 20 bp, Artificial sequence
  Synthetic Construct
catggggctcctctgtaagt

SEQ ID NO: 31 - DNA, 20 bp, Artificial sequence
  Synthetic Construct
gctgacgtgggagtactgtg

SEQ ID NO: 32 - DNA, 21 bp, Artificial sequence
  Synthetic Construct
ctgaggctggctgtactgtct

SEQ ID NO: 33 - DNA, 21 bp, Artificial sequence
  Synthetic Construct
tgacactggcaaaacaatgca

SEQ ID NO: 34 - DNA, 21 bp, Artificial sequence
  Synthetic Construct
ggtccttttcaccagcaagct

SEQ ID NO: 35 - DNA, 20 bp, Artificial sequence
  Synthetic Construct
gacggaggttgagatgaagc

SEQ ID NO: 36 - DNA, 20 bp, Artificial sequence
  Synthetic Construct
attcggggctctgtagtcct

SEQ ID NO: 37 - DNA, 25 bp, Artificial sequence
  Synthetic Construct
gcttcggcagcacatatactaaaat

SEQ ID NO: 38 - DNA, 21 bp, Artificial sequence
  Synthetic Construct
acgaatttgcgtgtcatcctt

SEQ ID NO: 39 - DNA, 23 bp, Artificial sequence
  Synthetic Construct
tcatgaagaagagtcctttcagc

SEQ ID NO: 40 - DNA, 21 bp, Artificial sequence
  Synthetic Construct
ggatgacgtaaagggataggg

SEQ ID NO: 41 - DNA, 23 bp, Artificial sequence
  Synthetic Construct
agaagggttgatttcaaacaaga

SEQ ID NO: 42 - DNA, 20 bp, Artificial sequence
  Synthetic Construct
ctccaaggcacgagtaaagg

SEQ ID NO: 43 - DNA, 20 bp, Artificial sequence
  Synthetic Construct
gagtggaccacactgcgcca

SEQ ID NO: 44 - DNA, 22 bp, Artificial sequence
  Synthetic Construct
tccacaaccctctgcacccagt

SEQ ID NO: 45 - DNA, 20 bp, Artificial sequence
  Synthetic Construct
acaacaacctacggcagaca

SEQ ID NO: 46 - DNA, 20 bp, Artificial sequence
  Synthetic Construct
ccacagacggtaatgtggtg

SEQ ID NO: 47 - DNA, 18 bp, Artificial sequence
  Synthetic Construct
gccaggactcccatgaaa

SEQ ID NO: 48 - DNA, 18 bp, Artificial sequence
  Synthetic Construct
gcttcaggtggcgcttac

SEQ ID NO: 49 - DNA, 19 bp, Artificial sequence
  Synthetic Construct
ggccatctcgcaacttaca

SEQ ID NO: 50 - DNA, 20 bp, Artificial sequence
  Synthetic Construct
gcagcacagacatgaaatcc

SEQ ID NO: 51 - DNA, 20 bp, Artificial sequence
  Synthetic Construct
ttctgatttgctgccttatc

SEQ ID NO: 52 - DNA, 23 bp, Artificial sequence
  Synthetic Construct
cttggattaacaggttgattact

SEQ ID NO: 53 - DNA, 21 bp, Artificial sequence
  Synthetic Construct
ggttgagtttaagccaatcca

SEQ ID NO: 54 - DNA, 20 bp, Artificial sequence
  Synthetic Construct
tgctgacctaggcttgatga

SEQ ID NO: 55 - DNA, 19 bp, Artificial sequence
  Synthetic Construct
agaggcactccaggagacg

SEQ ID NO: 56 - DNA, 20 bp, Artificial sequence
  Synthetic Construct
ctgaagggtccacacttgct

SEQ ID NO: 57 - DNA, 20 bp, Artificial sequence
  Synthetic Construct
aaggacgacaggcagagaga

SEQ ID NO: 58 - DNA, 22 bp, Artificial sequence
  Synthetic Construct
ttcttcatttaggcgttccttt

SEQ ID NO: 59 - DNA, 20 bp, Artificial sequence
  Synthetic Construct
gccttcggtccagttgcctt

SEQ ID NO: 60 - DNA, 21 bp, Artificial sequence
  Synthetic Construct
agtgcctctttgctgctttca

SEQ ID NO: 61 - DNA, 24 bp, Artificial sequence
  Synthetic Construct
aggctcagacaaatacttccatgc

SEQ ID NO: 62 - DNA, 24 bp, Artificial sequence
  Synthetic Construct
tctctggcatcgctgatcacttct

SEQ ID NO: 63 - DNA, 21 bp, Artificial sequence
  Synthetic Construct
aacctgtcaccatcgactgtg

SEQ ID NO: 64 - DNA, 21 bp, Artificial sequence
  Synthetic Construct
tgattgcgttcttccttacgg

SEQ ID NO: 65 - DNA, 19 bp, Artificial sequence
  Synthetic Construct
tcctggctcgacaaggagt

SEQ ID NO: 66 - DNA, 18 bp, Artificial sequence
  Synthetic Construct
ggtttcccaggccgtaag

SEQ ID NO: 67 - DNA, 22 bp, Artificial sequence
  Synthetic Construct
atatacctgaggtctgccagga

SEQ ID NO: 68 - DNA, 21 bp, Artificial sequence
  Synthetic Construct
atctgggtcaccgaaactgta

SEQ ID NO: 69 - RNA, 25 nt, Artificial sequence
  Synthetic Construct
caccguucccagauggggcgacggc

SEQ ID NO: 70 - RNA, 25 nt, Artificial sequence
  Synthetic Construct
caccguauauugcuaagacguugac